(19) United States
(12) Patent Application Publication
(10) Pub. No.: US 2015/0101132 A1
(43) Pub. Date: Apr. 16, 2015

(54) CATIONIC DYE BEARING AN ANIONIC ORGANIC COUNTERION, DYE COMPOSITION COMPRISING THEM AND PROCESS FOR DYEING KERATIN USING THESE DYES

(71) Applicant: L'OREAL, Paris (FR)

(72) Inventors: Herve David, La Varenne Saint Hilaire (FR); Annabelle Eudeline-Ly, Lamorlaye (FR)

(21) Appl. No.: 14/403,339

(22) PCT Filed: May 24, 2013
(86) PCT No.: PCT/EP2013/060769

§ 371 Date: Nov. 24, 2014

(30) Foreign Application Priority Data
May 24, 2012 (FR) 1254750

(51) Int. Cl. A61K 8/49; A61Q 5/10; C09B 23/16

(52) U.S. Cl. CPC A61K 8/4926; C09B 23/164; A61Q 5/10
USPC 8/426

(57) ABSTRACT

The invention relates to the dyeing of human keratin fibres, especially the hair, using cationic direct dyes bearing a particular sulf(on)ate, carboxylate, or phosph(on)ate counterion of formula (I) in which formula (I) Col⁽⁻⁾m, X⁻, m and n are as defined in the description. The invention relates to a dye composition comprising a cationic dye of formula (I), and to a process for dyeing keratin fibres such as the hair, using the said composition. Similarly, the invention relates to novel cationic compounds of formula (I) and to the uses thereof for dyeing keratin fibres. This composition makes it possible to obtain chromatic, powerful and particularly fast coloration on keratin fibres and particularly lightening coloration via an optical effect on dark hair.

Col⁽⁺⁾ₘ[X⁻]ₙ (I)

The invention relates to the dyeing of human keratin fibres using cationic direct dyes bearing a particular sulfate, sulfonate, carboxylate or phosphate counterion.

It is known practice to dye keratin fibres, especially the hair, by direct dyeing. The process conventionally used in direct dyeing consists in applying to keratin fibres direct dyes, which are coloured and colouring molecules that have affinity for the fibres, leaving them to diffuse and then rinsing the fibres.

The direct dyes that are conventionally used are, for example, dyes of the nitrobenzene type, anthraquinone dyes, nitropyridines and dyes of the azo, xanthene, acridine, azine or triarylmethane type.

These dyes may be anionic, cationic or neutral. Among the cationic direct dyes or “basic dyes” that are available in the field of dyeing keratin fibres, especially human keratin fibres, dyes bearing an exocyclic cationic charge of ammonium type, also known as Arianors, and others bearing endocyclic cationic charges, described in patent applications WO 95/01772, WO 95/15144 and EP-A-0 714 954, are already known. It is also known practice to use direct dyes combined with heat, including a direct dye (A) as defined below comprising an endocyclic cationic charge, and an anionic counterion bearing a long chain, to dye keratin fibres (FR2888746). All these dyes lead to colorations that have characteristics which are still insufficient, both as regards the homogeneity of the colour along the fibre (“unison”), in which case the coloration is said to be too selective, or as regards the fastness, in terms of the resistance to the various attacking factors to which the hair may be subjected, especially in terms of successive shampooing, light, and bad weather (see, for example, The chemistry of haircare products, JSDC, J. F. Corbett, 285-303 (1976), p. 290).

There is great demand for the lightening of the colour of keratin fibres, more particularly dark keratin fibres, towards lighter shades, optionally while modifying the shade thereof. Conventionally, to obtain a lighter coloration, a chemical bleaching process is performed. This process consists in treating the keratin materials, such as keratin fibres, especially the hair, with a strong oxidizing system, generally constituted by hydrogen peroxide optionally combined with persalts, usually in an alkaline medium.

This bleaching system has the drawback of degrading the fibres and of impairing their cosmetic properties. Specifically, the fibres have a tendency to become coarse, more difficult to disentangle and more brittle. Finally, the lightening or bleaching of keratin fibres with oxidizing agents is incompatible with treatments for changing the shape of the said fibres, particularly relaxing treatments.

Another lightening technique consists in applying to dark hair fluorescent direct dyes. This technique described especially in document FR 2830189 makes it possible to respect the quality of the keratin fibre during the treatment, but the fluorescent dyes used do not always have satisfactory shampoo-fastness and/or a satisfactory lightening effect.

The aim of the present invention is to provide novel dyes for human keratin fibres, such as the hair, which have improved dyeing properties, especially powerful, chromatic and/or remanent coloration of the hair with respect to external attacking factors, especially shampooing, without causing excessive staining of the scalp. The invention is also directed towards providing hair dyes which show little dyeing selectivity between the root and the end, which do not degrade keratin fibres and which do not impair their cosmetic properties.

These aims are achieved with the present invention, one subject of which is a process for dyeing and/or lightening keratin fibres, especially dark keratin fibres, using at least one dye of formula (I) below:

Col⁽⁺⁾ₘ[X⁻]ₙ (I)

and also the optical isomers and geometrical isomers thereof, and solvates thereof such as hydrates;
in which formula (I):
—X⁻, which may be identical or different, represent a counterion chosen from: the following monoanionic counterions: carboxylates i) Rₐ—CO₂⁻; sulfonates ii) R_{b}—SO₃⁻; sulfates iii) R_{c}—O—SO₃⁻; phosphates iv) R_{d}—O—P(O)(OH)O⁻ or R_{d}—O—PO₃²⁻; phosphate esters v) Rₑ—P(OR_{f})O₂⁻; the following polyanionic counterions: vi) L-(X′⁻)ₓ; vii) anionic oligomers comprising at least one anionic group X′⁻ other than vi); viii) anionic polymers comprising at least one anionic group X′⁻ other than vi); and ix) a mixture thereof; in which formulae i) to viii):
X′⁻, which may be identical or different, represent a group CO₂⁻, SO₃⁻, O—SOs₃⁻, O—PO₃²⁻ or P(OR_{f})O₂⁻; x represents an integer between 2 and 20 inclusive, in particular between 2 and 10, and preferentially x=2 or 3; and L represents:
a) a linear or branched C₁-C₄₀ and preferably C₂-C₃₀ divalent or polyvalent hydrocarbon-based chain, which is optionally substituted, preferably with at least one hydroxyl group, the said chain being saturated or unsaturated, optionally interrupted and/or terminated at least one of its ends with one or more divalent groups or a combination thereof chosen from:
   —N(R^{a})—; —O—; —S—; —S(O)—, —S(O)₂—, —C(O)— and —C(S)— with R^{a} being chosen from a hydrogen atom and a C₁-C₄ alkyl, hydroxy(C₁-C₈)alkyl or amino(C₁-C₈)alkyl radical;
   an aromatic or non-aromatic, saturated or unsaturated, fused or non-fused (hetero)cyclic radical, optionally comprising one or more identical or different, optionally substituted heteroatoms, preferably phenylene; and
      preferentially, the divalent group(s) or combinations thereof are chosen from —S—, —O—; —N(Rₐ)—; —C(O)— with Rₐ chosen from a hydrogen atom and a C₁-C₄ alkyl radical;
b) an aromatic or non-aromatic, saturated or unsaturated, optionally substituted, fused or non-fused divalent or polyvalent (hetero)cyclic group, optionally comprising one or more identical or different, optionally substituted heteroatoms, preferably arylene;
Rₐ, R_{b} and R_{c} represent a radical chosen from: i) optionally substituted linear or branched (C₄-C₄₀)alkyl; ii) optionally substituted linear or branched (C₄-C₄₀)alkenyl; iii) optionally substituted heteroaryl; iv) optionally substituted (hetero)cycloalkyl; v) optionally substituted linear or branched (hetero)aryl(C₁-C₄)alkyl; vi) optionally substituted (hetero)cycloalkyl(C₁-C₄)alkyl; the said alkyl or alkenyl group i) or ii) possibly being interrupted and/or terminated with one or more identical or different divalent (hetero)aryls, divalent (hetero)cycloalkyls and heteroatoms chosen from an oxygen or sulfur atom, an amino —N(R)—, —C(O)— or —C(S)— group or a combination thereof, with R representing a hydrogen atom or a (C₁-C₆)alkyl group;
Rₐ and R_{c} may also represent vii) an optionally substituted aryl group;
R_{d}, Rₑ and R_{f}, which may be identical or different, represent a radical chosen from: i) optionally substituted linear or branched (C₁-C₄₀)alkyl; ii) optionally substituted linear or branched (C₂-C₄₀)alkenyl; iii) optionally substituted (hetero)aryl; iv) optionally substituted (hetero)cycloalkyl; v) optionally substituted linear or branched (hetero)aryl(C₁-C₄)alkyl; vi) optionally substituted (hetero)cycloalkyl(C₁-C₄)alkyl; the said alkyl or alkenyl group i) or ii) possibly being interrupted and/or terminated with one or more identical or different divalent (hetero)aryls, divalent (hetero)cycloalkyls and heteroatoms chosen from an oxygen or sulfur atom, an amino —N(R)—, —C(O)— or —C(S)— group or a combination thereof, with R representing a hydrogen atom or a (C₁-C₆)alkyl group;
Col⁽⁺⁾ₘ represents the cationic part of a cationic direct dye or “basic” dye comprising m cationic charge(s) comprising:
   at least one heteroaryl bearing an endocyclic cationic charge, i.e. intrinsic to the structure of the dye; and/or
   at least one (hetero)aryl or (hetero)cycloalkyl group bearing an exocyclic cationic charge;
      the said cationic charges being chosen from:
a) the ammoniums of formula (1) below:

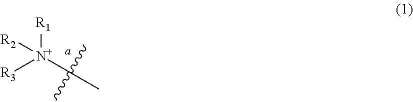

b) optionally substituted heteroaromatic groups bearing exocyclic or endocyclic, preferably endocyclic, cationic charge(s), such as (benz)imidazolium, indolinium, (benzo)triazolium, (benzo)pyrylium and (benzo)pyridinium groups, especially of formulae (2) and (2′) below:

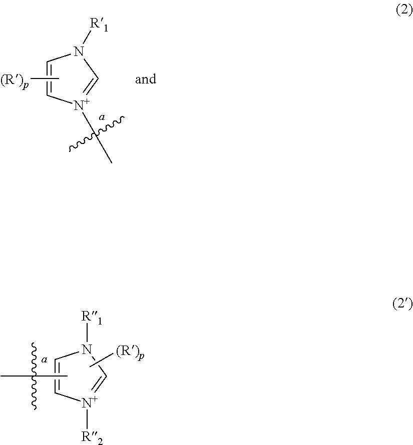

in which formulae (1), (2) and (2′):
   R₁, R₂ and R₃, which may be identical or different, represent a group chosen from: i) linear or branched (C₁-C₂₀)alkyl; ii) (C₂-C₂₀)alkenyl; iii) (hetero)aryl(C₁-C₂₀)alkyl; (hetero)cycloalkyl(C₁-C₂₀)alkyl or iv) (hetero)aryl; the alkyl or alkenyl group of the groups of i), ii), iii) or iv) possibly being a) interrupted with one or more identical or different heteroatoms chosen from an oxygen or sulfur atom, an amino —N(R)—, ammonium —N⁺(Rₐ)(R_{b})—, —C(O)— or —C(S)— group or a combination thereof, with R, Rₐ and R_{b}, which may be identical or different, representing a hydrogen atom or an alkyl group and/or b) substituted especially with at least one hydroxyl, hydroxycarbonyl or carboxyl, (C₁-C₆)alkoxycarbonyl, alkyl(C₁-C₆)carbonyloxy, carbamoyloxy, (di)(C₁-C₆)(alkyl)silyl or tri(C₁-C₅)alkylsilyl group; preferentially, R₁, R₂ and R₃ represent a linear (C₁-C₁₀)alkyl group substituted with at least one hydroxyl group, a hydroxycarbonyl group or an alkyl(C₁-C₂)carbonyloxy, carbamoyloxy or tri(C₁-C₂)alkylsilyl group; preferentially, R₁, R₂ and R₃ represent a linear (C₁-C₁₀)alkyl group substituted with at least one hydroxyl group, a hydroxycarbonyl group or an alkyl(C₁-C₂)carbonyloxy, carbamoyloxy or tri(C₁-C₂)alkylsilyl group;
   or alternatively the radicals R₁ and R₂ form, together with the quaternized nitrogen atom, a saturated 5- or 6-membered heterocycle, the said heterocycle being optionally partially unsaturated, optionally interrupted with a heteroatom chosen from the oxygen atom, the group —N(R)—, or ammonium —N⁺(R′ₐ)(R′_{b})— with R, R′ₐ and R′_{b} as defined previously and/or the said heterocycle possibly being substituted with one or more groups such as (C₁-C₆)alkyl; preferentially, R₁ and R₂ are borne by a quaternized nitrogen atom, and in particular they form, together with the quaternized nitrogen atom, a pyrrolidinium, morpholinium, piperazinium or piperidinium group;
   R′₁, R″₁ and R″₂, which may be identical or different, represent a group chosen from i) linear or branched (C₁-C₂₀)alkyl; ii) (C₂-C₂₀)alkenyl; the alkyl or alkenyl group of the groups of i) and ii) possibly being interrupted with one or more identical or different heteroatoms chosen from an oxygen or sulfur atom or ammonium —N⁺(R′ₐ)(R′_{b})—, —C(O)—, —C(S)— or a combination thereof, with R, R′ₐ and R′_{b} as defined previously;
   R′₁, R″₁, and R″₂, which may be substituted with at least one hydroxyl, hydroxycarbonyl or carboxyl, (C₁-C₆)alkoxycarbonyl, alkyl(C₁-C₆)carbonyloxy, carbamoyloxy, (di)(C₁-C₆)(alkyl)silyl or tri(C₁-C₅)alkylsilyl group; preferentially, R′₁, R″₁, and R″₂ represent a linear or branched (C₁-C₁₀)alkyl group optionally substituted with at least one hydroxyl group, a hydroxycarbonyl group or a (C₁-C₂)alkylcarbonyloxy, carbamoyloxy or tri(C₁-C₂)alkylsilyl group;
   R′ represents a group chosen from i) (C₁-C₅)alkyl, ii) (C₁-C₅)alkoxycarbonyl and iii) hydroxyl;
   a represents the linker arm connecting the cationic groups of formulae (1), (2) and/or (2′) to the dye or alternatively to an optionally substituted alkyl radical;
   p is 0, 1, 2 or 3, it being understood that when p is 2 or 3, the radicals R′ are identical or different, or two radicals borne by contiguous carbon atoms together form a fused (hetero)aryl group such as benzo;
m and n, which may be identical or different, represent an integer between 1 and 50 inclusive;
   it being understood that:
when the cationic part of the cationic direct dye contains a cationic group intrinsic to the structure or a cationic group of formula (1), (2) or (2′), then m=n=1; and
when the cationic part of the cationic direct dye contains cationic groups other than the endocyclic cationic group, it is combined with one or more organic anionic counterion(s) X⁻ for affording electrical neutrality to formula (I);
the compound of formula (I) is other than compound (A) below:

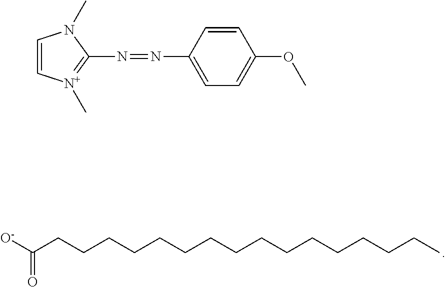 [SMILES: COc1ccc(N=Nc2n(C)cc[n+]2C)cc1 ; CCCCCCCCCCCCCCCCC(=O)[O-]]

Another subject of the invention is a dye composition comprising, in a suitable cosmetic medium, at least one cationic dye of formula (I) as defined previously, it being understood that the dye of formula (I) cannot represent compounds (A) and (B) below:

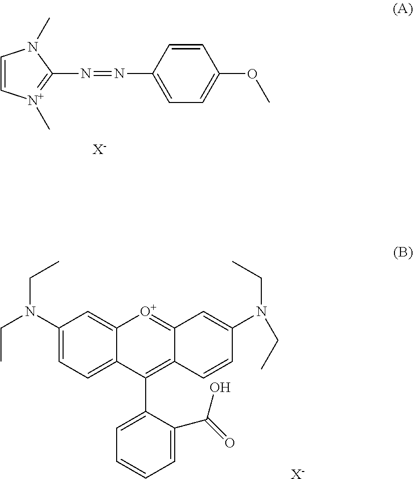 [SMILES: COc1ccc(N=Nc2n(C)cc[n+]2C)cc1 ; "X-" ; CCN(CC)c1ccc2c(-c3ccccc3C(=O)O)c3ccc(N(CC)CC)cc3[o+]c2c1 ; "X-"]

with X⁻ representing:

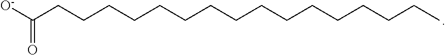 [SMILES: CCCCCCCCCCCCCCCCC(=O)[O-]]

Another subject of the invention concerns a cationic dye of formula (I) as defined previously for dyeing keratin fibres such as the hair, it being understood that the dye of formula (I) cannot represent compounds (A) and (B) as defined previously or compounds (C) and (D) below:

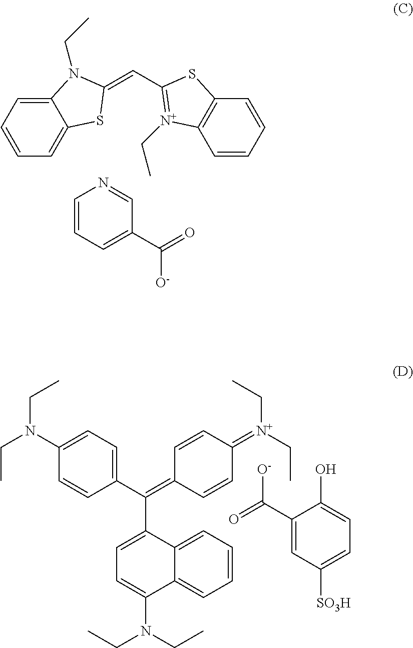 [SMILES: CCN1/C(=C/c2sc3ccccc3[n+]2CC)Sc2ccccc21 ; O=C([O-])c1cccnc1 ; CCN(CC)c1ccc(C(=C2C=CC(=[N+](CC)CC)C=C2)c2ccc(N(CC)CC)c3ccccc23)cc1 ; O=C([O-])c1cc(S(=O)(=O)O)ccc1O]

Another subject of the invention concerns the use of at least one cationic dye of formula (I) as defined previously for dyeing keratin fibres such as the hair.

Another subject of the invention concerns the use of at least one fluorescent cationic dye of formula (I) as defined previously, particularly in the orange range, for optically lightening dark keratin fibres such as hair with a tone depth of less than or equal to 6 and preferentially less than or equal to 4, even in the absence of a chemical oxidizing agent other than atmospheric oxygen.

With the dyes of the invention, it is possible to improve the dyeing properties of cationic dyes especially in terms of chromaticity, power and fastness by replacing the “standard” anionic counterions such as halide, mesylate or tosylate anions with one or more organic or mineral (poly)anions X⁻ as defined previously. It has also been found that the optical lightening can be improved by using dye(s) of formula (I) as defined previously.

The cationic compounds of formula (I) according to the invention are moreover stable with respect to oxidizing agents, and show satisfactory solubility cosmetic dyeing media.

Thus, the compounds of formula (I) make it possible especially to significantly improve the remanence, in particular with respect to shampooing, and/or the optical lightening of dark keratin fibres.

For the purposes of the present invention, and unless otherwise indicated:
The term “dark keratin fibres”, especially dark hair, means fibres which are naturally or artificially dark and whose tone depth is less than or equal to 6 (dark blond) and preferably less than or equal to 4 (chestnut-brown). The notion of “tone” is based on the classification of natural shades, one tone separating each shade from the shade immediately following or preceding it. This definition and the classification of natural shades are well known to hairstyling professionals and are published in the book Science des traitements capillaires [Hair treatment sciences] by Charles Zviak, 1988, published by Masson, pp. 215 and 278. The tone depths range from 1 (black) to 10 (very light blond), one unit corresponding to one tone; the higher the figure, the lighter the shade.
a “hydrocarbon-based chain” is “unsaturated” when it comprises one or more double bonds and/or one or more triple bonds;
the “aryl” or “heteroaryl” radicals or the aryl or heteroaryl part of a radical may be substituted with at least one substituent borne by a carbon atom, chosen from:
   a C₁-C₁₆ and preferably C₁-C₈ alkyl radical optionally substituted with one or more radicals chosen from hydroxyl, C₁-C₂ alkoxy, C₂-C₄ (poly)hydroxyalkoxy, acylamino, amino substituted with two C₁-C₄ alkyl radicals, which may be identical or different, optionally bearing at least one hydroxyl group, or the two radicals possibly forming, with the nitrogen atom to which they are attached, a saturated or unsaturated, optionally substituted 5- to 7-membered and preferably 5- or 6-membered heterocycle optionally comprising another nitrogen or non-nitrogen heteroatom;
   a halogen atom such as chlorine, fluorine or bromine;
   a hydroxyl group;
   a C₁-C₂ alkoxy radical;
   a C₂-C₄ (poly)hydroxyalkoxy radical;
   an amino radical;
   nitro;
   a 5- or 6-membered heterocycloalkyl radical;
   an optionally cationic 5- or 6-membered heteroaryl radical, preferentially imidazolium, optionally substituted with a (C₁-C₄)alkyl radical, preferentially methyl;
   an amino radical substituted with one or two identical or different C₁-C₆ alkyl radicals, optionally bearing at least:
   i) a hydroxyl group;
   ii) one amino group optionally substituted with one or two optionally substituted C₁-C₃ alkyl radicals, it being possible for said alkyl radicals to form, with the nitrogen atom to which they are attached, a saturated or unsaturated and optionally substituted 5- to 7-membered heterocycle
   optionally comprising at least one other nitrogen or non-nitrogen heteroatom,
   an acylamino radical (—N(R)—C(O)R′) in which the radical R is a hydrogen atom or a C₁-C₄ alkyl radical optionally bearing at least one hydroxyl group and the radical R′ is a C₁-C₂ alkyl radical;
   a carbamoyl radical ((R)₂N—C(O)—) in which the radicals R, which may be identical or different, represent a hydrogen atom or a C₁-C₄ alkyl radical optionally bearing at least one hydroxyl group;
   a carboxylic acid or ester radical, (—O—C(O)R′) or (—C(O)OR′), in which the radical R′ is a hydrogen atom or a C₁-C₄ alkyl radical optionally bearing at least one hydroxyl group, and the radical R′ is a C₁-C₂ alkyl radical;
   the carboxylic radical possibly being in acid or salified form (preferably with an alkali metal or a substituted or unsubstituted ammonium);
   an alkylsulfonylamino radical (R′S(O)₂—N(R)—) in which the radical R represents a hydrogen atom or a C₁-C₄ alkyl radical optionally bearing at least one hydroxyl group and the radical R′ represents a C₁-C₄ alkyl radical, or a phenyl radical;
   an aminosulfonyl radical ((R)₂N—S(O)₂—) in which the radicals R, which may be identical or different, represent a hydrogen atom or a C₁-C₄ alkyl radical optionally bearing at least one hydroxyl group,
   a cyano group (CN);
   a (poly)haloalkyl group, preferentially trifluoromethyl (CF₃);
the cyclic or heterocyclic part of a non-aromatic radical may be substituted with at least one substituent borne by a carbon atom, chosen from the groups:
   hydroxyl,
   C₁-C₄ alkoxy or C₂-C₄ (poly)hydroxyalkoxy,
   alkylcarbonylamino ((RC(O)—NR′—) in which the radical R′ is a hydrogen atom or a C₁-C₄ alkyl radical optionally bearing at least one hydroxyl group, and the radical R is a C₁-C₂ alkyl radical or an amino radical substituted with two C₁-C₄ alkyl groups, which may be identical or different, optionally bearing at least one hydroxyl group, the said alkyl radicals possibly forming, with the nitrogen atom to which they are attached, a saturated or unsaturated, optionally substituted 5- to 7-membered heterocycle optionally comprising at least one other nitrogen or non-nitrogen heteroatom;
   alkylcarbonyloxy ((RC(O)—O—) in which the radical R is a C₁-C₄ alkyl radical or an amino radical substituted with two identical or different C₁-C₄ alkyl groups optionally bearing at least one hydroxyl group, said alkyl radicals possibly forming with the nitrogen atom to which they are attached a saturated or unsaturated, optionally substituted 5- to 7-membered heterocycle, optionally comprising at least one other nitrogen or non-nitrogen heteroatom;
   alkoxycarbonyl ((RO—C(O)—) in which the radical R is a C₁-C₄ alkyl radical or an amino radical substituted with two identical or different C₁-C₄ alkyl groups optionally bearing at least one hydroxyl group, said alkyl radicals possibly forming with the nitrogen atom to which they are attached a saturated or unsaturated, optionally substituted 5- to 7-membered heterocycle, optionally comprising at least one other nitrogen or non-nitrogen heteroatom;
a cyclic or heterocyclic radical, or a non-aromatic part of an aryl or heteroaryl radical, may also be substituted with one or more oxo groups;
an “aryl” radical represents a fused or non-fused monocyclic or polycyclic group containing from 6 to 22 carbon atoms, and in which at least one ring is aromatic; in particular, the aryl radical is a phenyl, biphenyl, naphthyl, indenyl, anthracenyl or tetrahydronaphthyl and more preferentially phenyl or tetrahydronaphthyl;
a “heteroaryl” radical represents a 5- to 22-membered, monocyclic or polycyclic fused or non-fused group, comprising from 1 to 6 heteroatoms chosen from a nitrogen, oxygen, sulfur and selenium atom, at least one ring of which is aromatic; preferentially, a heteroaryl radical is chosen from acridinyl, benzimidazolyl, benzobistriazolyl, benzopyrazolyl, benzopyridazinyl, benzoquinolyl, benzothiazolyl, benzotriazolyl, benzoxazolyl, pyridinyl, tetrazolyl, dihydrothiazolyl, imidazopyridyl, imidazolyl, indolyl, isoquinolyl, naphthoimidazolyl, naphthoxazolyl, naphthopyrazolyl, oxadiazolyl, oxazolyl, oxazolopyridyl, phenazinyl, phenoxazolyl, pyrazinyl, pyrazolyl, pyrilyl, pyrazoyltriazyl, pyridyl, pyridinoimidazolyl, pyrrolyl, quinolyl, tetrazolyl, thiadiazolyl, thiazolyl, thiazolopyridinyl, thiazoylimidazolyl, thiopyrylyl, triazolyl, xanthyl and the ammonium salt thereof;
a “cyclic” radical is a “cycloalkyl” radical, i.e. a non-aromatic, monocyclic or polycyclic, fused or non-fused radical, containing from 5 to 22 carbon atoms, which may comprise one or more unsaturations, such as cyclohexyl or cyclopentyl;
a “heterocyclic” radical is a non-aromatic, monocyclic or polycyclic, fused or non-fused 5- to 22-membered radical, comprising from 1 to 6 heteroatoms chosen from nitrogen, oxygen, sulfur and selenium atoms, morpholinyl, thiomorpholinyl, piperidyl, piperazinyl, pyrrolidinyl, tetrahydrofuryl, tetrahydrothiophenyl, azepanyl, thioazepanyl; preferentially pyrrolidinyl and morpholino;
a “cationic heteroaryl radical” is a heteroaryl group as defined previously, which comprises an endocyclic or exocyclic cationic group;
   when the charge is endocyclic, it is included in the electron delocalization via the mesomeric effect, for example it is a pyridinium, imidazolium or indolinium group;

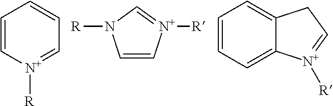

with R and R′ being a heteroaryl substituent as defined previously and particularly a (hydroxy)(C₁-C₈)alkyl group such as methyl;
   when the charge is exocyclic, it is not included in the electron delocalization via the mesomeric effect, for example it is an ammonium or phosphonium substituent R⁺ such as trimethylammonium, which is outside the heteroaryl such as pyridyl, indolyl, imidazolyl or naphthalimidyl in question;

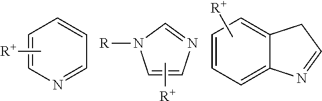

with R being a heteroaryl substituent as defined previously and R⁺ an ammonium RₐR_{b}R_{c}N⁺—, phosphonium RₐR_{b}R_{c}P⁺- or ammonium RₐR_{b}R_{c}N⁺—(C₁-C₆)alkylamino group with Rₐ, R_{b} and R_{c}, which may be identical or different, representing a hydrogen atom or a group (C₁-C₈)alkyl such as methyl;
an “alkyl” radical is a linear or branched C₁-C₁₆ and preferably C₁-C₈ hydrocarbon-based radical; particularly C₁-C₄ such as methyl or ethyl;
an “alkeny” radical is a linear or branched C₂-C₂₀ hydrocarbon-based radical comprising one or more conjugated or unconjugated double bonds, in particular a C₄-C₁₀ radical comprising one, two or three double bonds, preferentially only one double bond;
the term “optionally substituted” attributed to the alkyl radical means that the said alkyl radical may be substituted with one or more radicals chosen from the following radicals: i) hydroxyl, ii) C₁-C₄ alkoxy, iii) acylamino, iv) amino optionally substituted with one or two identical or different C₁-C₄ alkyl radicals, the said alkyl radicals possibly forming, with the nitrogen atom that bears them, a 5- to 7-membered heterocycle, optionally comprising another nitrogen or non-nitrogen heteroatom, v) phenyl, vi) (C₁-C₆)alkoxycarbonyl, vii) (C₁-C₆)alkylcarbonyloxy, viii) H—C(O)—O—;
an “alkoxy” radical is an alkyl-oxy or alkyl-O— radical for which the alkyl radical is a linear or branched C₁-C₁₆ and preferentially C₁-C₈ hydrocarbon-based radical; particularly C₁-C₄ such as methoxy or ethoxy, and when the alkoxy group is optionally substituted, this means that the alkyl group is optionally substituted as defined above;
a “(poly)haloalkyl” radical is an “alkyl” radical as defined previously, in which one or more hydrogen atoms are replaced with one or more halogen atoms such as the fluorine, chlorine or bromine atom; a polyhaloalkyl that may be mentioned is the trifluoromethyl group;
an “alkylthio” radical is a radical alkyl-S— for which the alkyl radical is a linear or branched C₁-C₁₆ and preferentially C₁-C₈ hydrocarbon-based radical; particularly C₁-C₄ such as methylthio or ethylthio, and when the alkylthio group is optionally substituted, this means that the alkyl group is optionally substituted as defined above;
an anionic counterion is organic or mineral, preferentially chosen from halide anions such as Cl⁻, Br⁻ or I⁻, and organic anions such as mesylates;
when the expression “at least one” is used, “one or more” is implied.

Furthermore, unless otherwise indicated, the limits delimiting the extent of a range of values are included in this range of values.

According to the present invention, the term “dye” means a compound which has the capacity of colouring and which is in the form of a coloured compound that may be observed with the naked eye (absorbing light at a wavelength in the UV and visible radiation range, i.e. at a wavelength λ_{abs} of between 250 and 800 nm, particularly in the visible spectrum between 400 and 700 nm.

The term “fluorescent dye” means a dye as defined previously, which, besides the fact that it is coloured, is fluorescent, i.e. it has the capacity of re-emitting at least part of the absorbed light, preferably at least the majority of the absorbed light, in the visible region at a wavelength higher than the absorbed wavelength. In particular, the fluorescent dye is capable of absorbing UV or visible radiation at a wavelength X_{abs} of between 250 and 800 nm and capable of re-emitting in the visible range at an emission wavelength λₑₘ of between 400 and 800 nm. Preferably, the fluorescent dye is a dye in the orange, violet, blue and green range. More preferentially, the fluorescent dye(s) (I) are dyes in the orange range.

I. Dyes of formula (I)

### The Anionic Counterion X⁻

According to a preferred embodiment of the invention, X⁻ represents a monocarboxylate belonging to formula i) Rₐ—CO₂ as defined previously, preferably with Rₐ representing a C₄-C₂₀ alkyl or C₄-C₂₀ alkenyl group comprising from 1 to 4 unsaturations, the alkyl or alkenyl group being optionally substituted with at least one hydroxyl or aryl group such as phenyl and/or optionally interrupted with a group C(O). More particularly, X⁻ is chosen from the monocarboxylates 1 to 19 below, and also optical or geometrical isomers thereof:

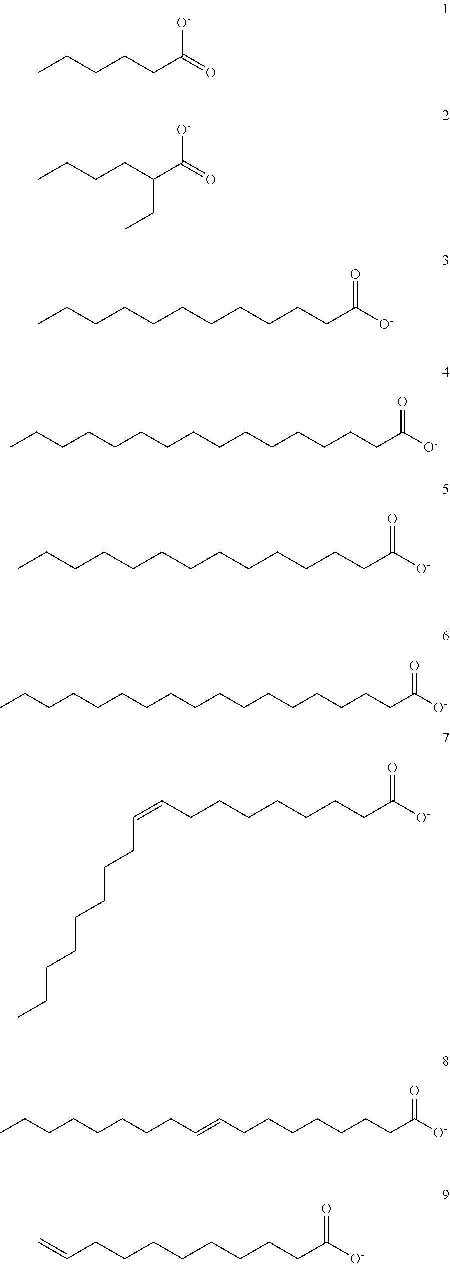

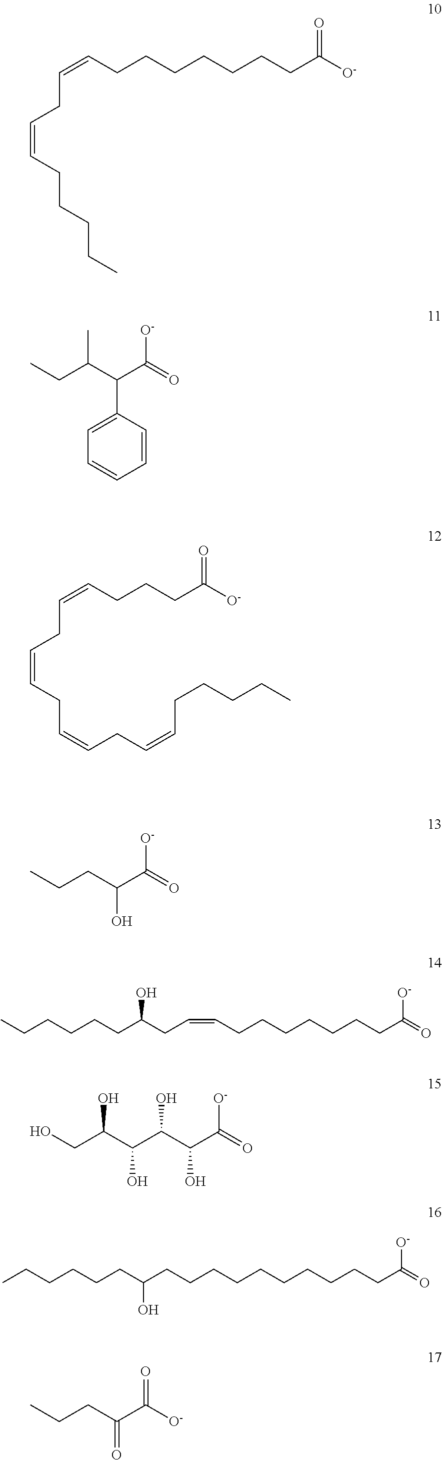

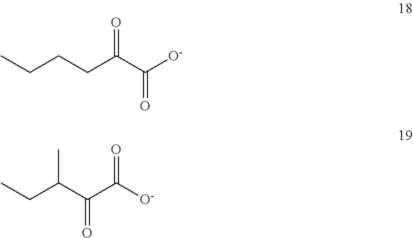

According to a preferred embodiment of the invention, X⁻ represents a dicarboxylate belonging to the formula vi) L-(X′⁻)ₓ with x=2 and X′⁻═CO₂⁻ as defined previously, and more particularly L represents a linear or branched C₂-C₆ alkylene group, optionally substituted with at least one hydroxyl or aryl group such as phenyl, and/or optionally interrupted with a group C(O). More particularly, X⁻ is chosen from the dicarboxylates 20 to 22 below, and also optical or geometrical isomers thereof:

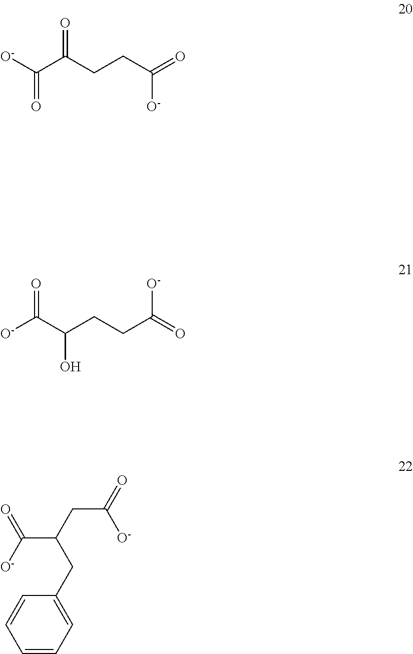 [SMILES: O=C([O-])CCC(=O)C(=O)[O-] ; O=C([O-])CCC(O)C(=O)[O-] ; O=C([O-])CC(Cc1ccccc1)C(=O)[O-]]

According to a preferred embodiment of the invention, X⁻ represents a tricarboxylate belonging to the formula vi) L-(X′⁻)ₓ with x=3 and X′⁻═CO₂⁻ as defined previously, and more particularly L represents a linear or branched C₃-C₆ alkylene group, optionally substituted with at least one hydroxyl group. More particularly, X⁻ is chosen from the tricarboxylates 23 and 24 below, and also optical or geometrical isomers thereof:

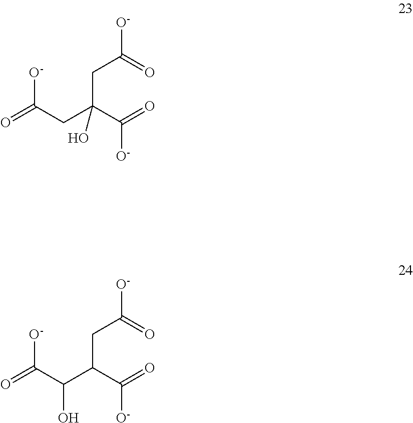 [SMILES: O=C([O-])CC(O)(CC(=O)[O-])C(=O)[O-] ; O=C([O-])CC(C(=O)[O-])C(O)C(=O)[O-]]

According to a preferred embodiment of the invention, X⁻ represents a monosulfonate belonging to formula ii) R_{b}—SO₃⁻ as defined previously, and preferably with R_{b} representing a linear or branched C₆-C₃₀ alkyl or linear or branched C₆-C₃₀ alkenyl group comprising from 1 to 2 unsaturations, the alkyl or alkenyl group being optionally substituted with at least one hydroxyl or (C₁-C₄)alkoxycarbonyl group, and in particular the hydroxyl and (C₁-C₄)alkoxycarbonyl groups being alpha to the sulfonate group. More particularly, X⁻ is chosen from the monosulfonates 25 to 46 below, and also optical or geometrical isomers thereof:

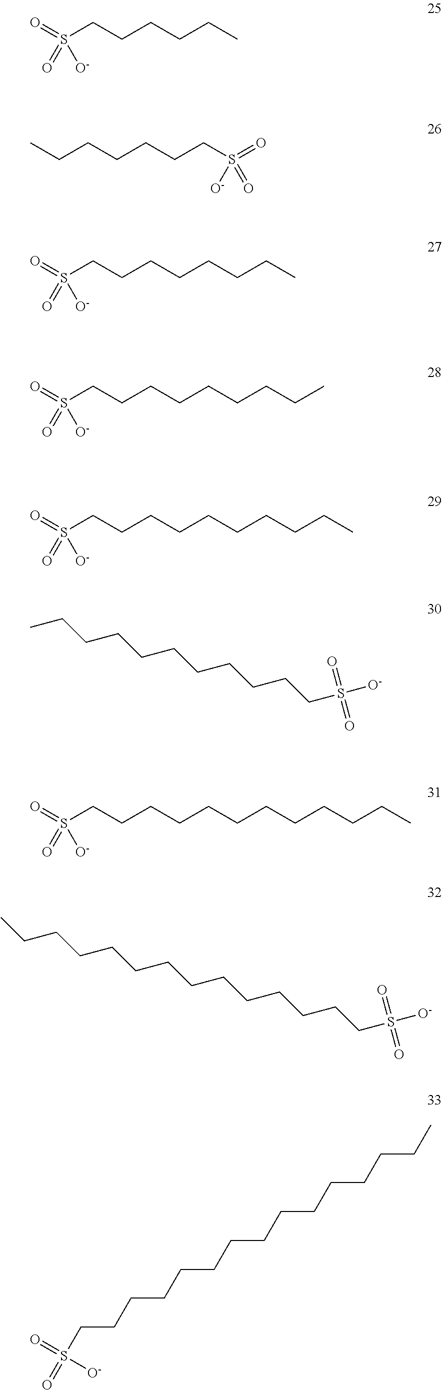

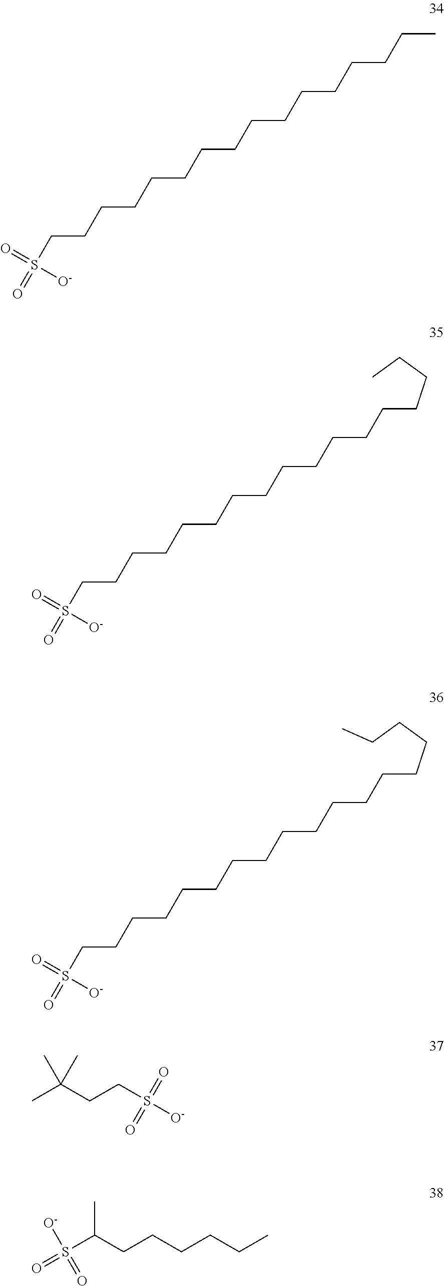

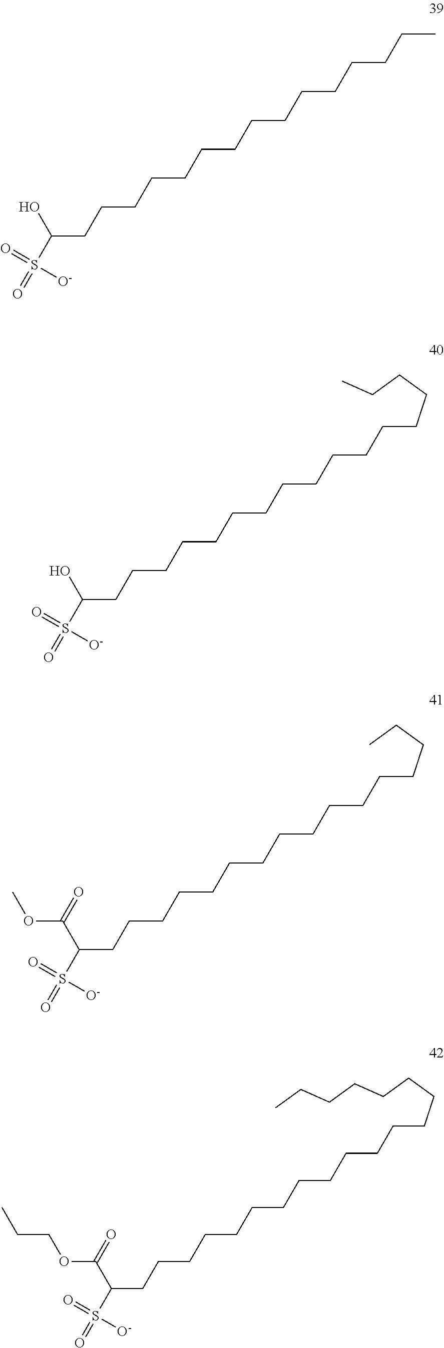

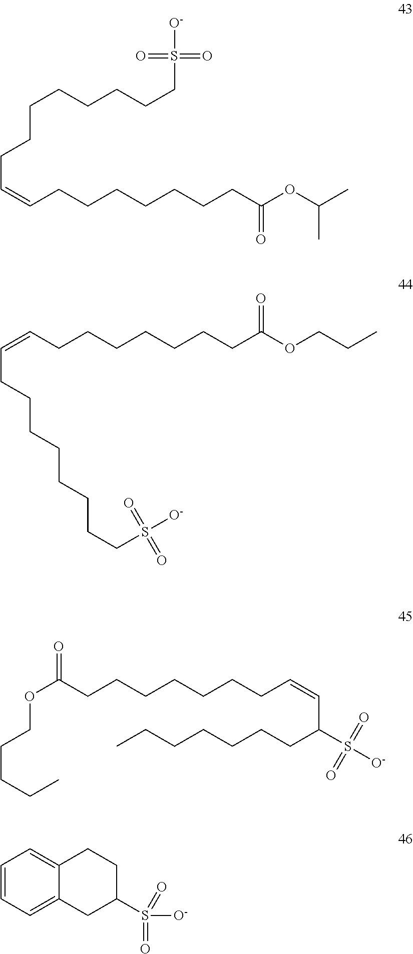 [SMILES: CC(C)OC(=O)CCCCCCC/C=C\CCCCCCCCS(=O)(=O)[O-] ; CCCOC(=O)CCCCCCC/C=C\CCCCCCCCS(=O)(=O)[O-] ; CCCCCCCC(/C=C\CCCCCCCC(=O)OCCCCC)S(=O)(=O)[O-] ; O=S(=O)([O-])C1CCc2ccccc2C1]

According to a preferred embodiment of the invention, X⁻ represents a disulfonate belonging to the formula vi) L-(X′⁻)ₓ with x=2 and X′⁻═SO₃⁻ as defined previously, and more particularly L represents a linear or branched C₄-C₄₀ alkylene group, optionally substituted with at least one hydroxyl group, preferably located alpha to the sulfonate groups; and/or optionally interrupted with one or more oxygen atoms —O—.

According to one particular mode of the invention, the radical L is a divalent group —(CH₂)ₙ—(CH₂—CH₂—O)ₘ—(CH₂)_{q}— with n, m and q, which may be identical or different, being integers between 0 and 20 inclusive, and the sum n+m+q being between 4 and 40 inclusive. More particularly, X⁻ is chosen from the disulfonates 47 to 50 below:

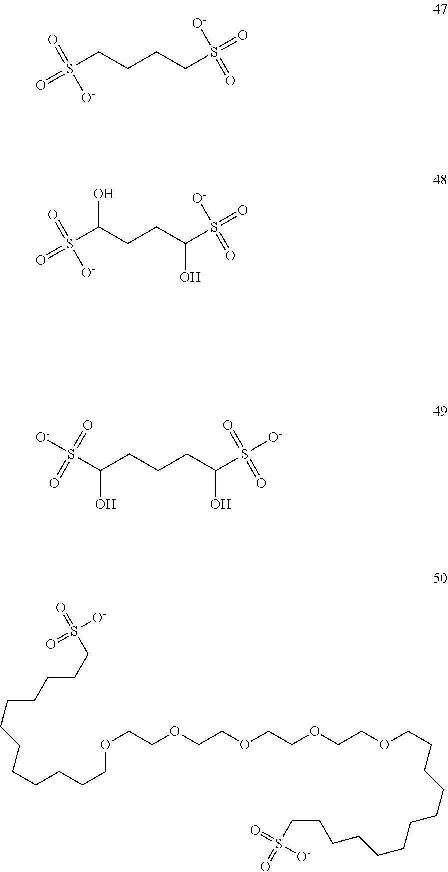 [SMILES: O=S(=O)([O-])CCCCS(=O)(=O)[O-] ; O=S(=O)([O-])C(O)CCC(O)S(=O)(=O)[O-] ; O=S(=O)([O-])C(O)CCCC(O)S(=O)(=O)[O-] ; O=S(=O)([O-])CCCCCCCCCCCCOCCOCCOCCOCCOCCCCCCCCCCCCS(=O)(=O)[O-]]

According to a preferred embodiment of the invention, X⁻ represents a monosulfonate belonging to formula iii) R_{c}—O—SO₃⁻ as defined previously, and preferably with R_{c} representing a linear or branched C₆-C₃₀ alkyl or linear or branched C₆-C₃₀ alkenyl group comprising from 1 to 2 unsaturations, the alkyl or alkenyl group being optionally substituted with at least one hydroxyl group; or alternatively R_{c} represents a (hetero)cycloalkyl(C₁-C₄)alkyl group optionally substituted with one or more hydroxyl or (C₁-C₄)alkyl groups. Preferentially, R_{c} represents a (hetero)cycloalkyl(C₁-C₄)alkyl group chosen from a 5- or 6-membered cycloalkyl or heterocycloalkyl group such as cyclohexyl, pyranose or furanose. More particularly, X⁻ is chosen from the monosulfonates 51 to 70 below, and also optical or geometrical isomers thereof:

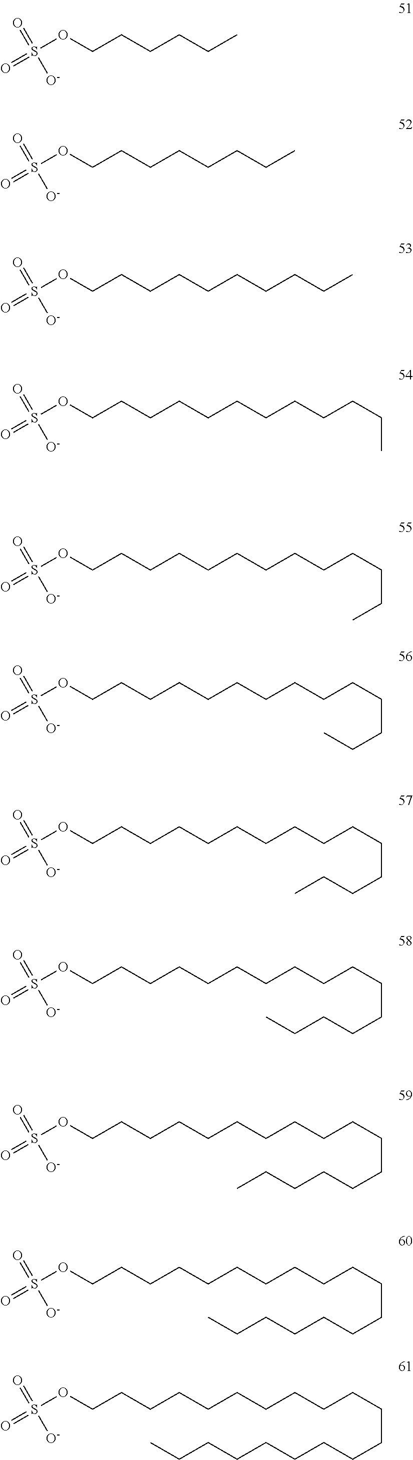

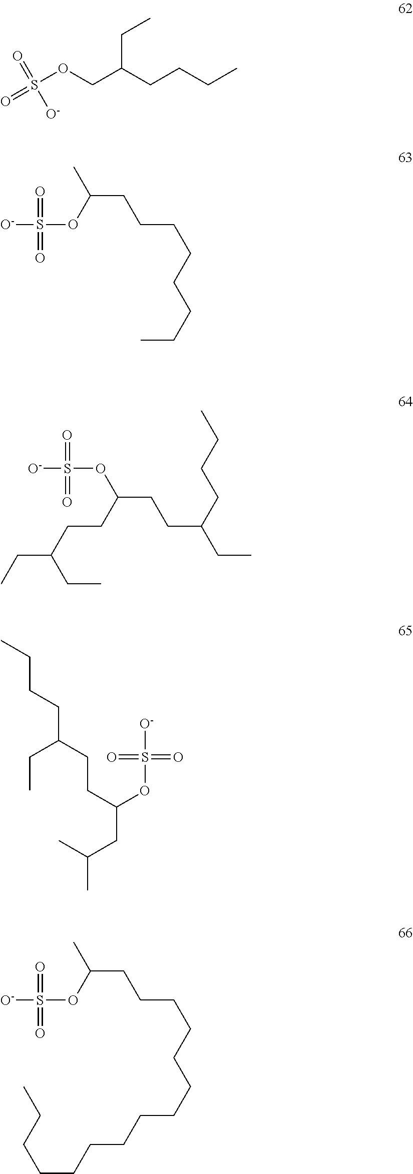

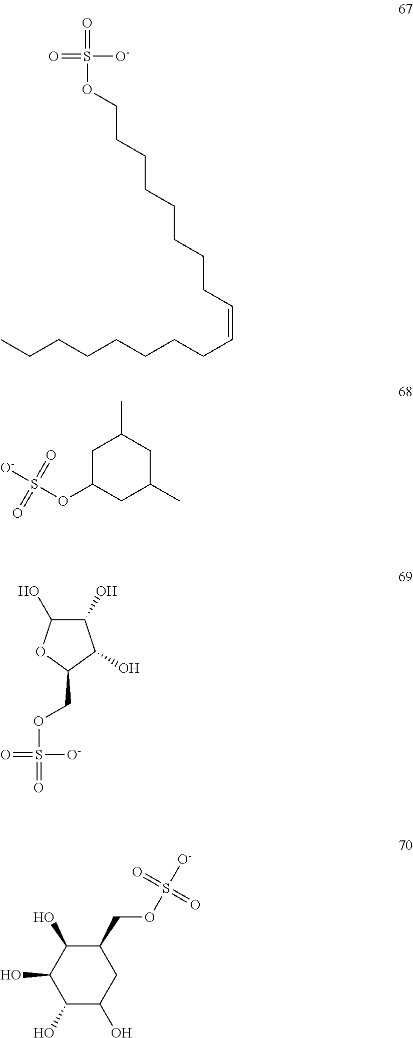 [SMILES: CCCCCCCC/C=C\CCCCCCCCOS(=O)(=O)[O-] ; CC1CC(C)CC(OS(=O)(=O)[O-])C1 ; O=S(=O)([O-])OC[C@H]1OC(O)[C@H](O)[C@@H]1O ; O=S(=O)([O-])OC[C@H]1CC(O)[C@H](O)[C@@H](O)[C@H]1O]

According to a preferred embodiment of the invention, X⁻ represents a sulfate/sulfonate mixed counterion belonging to the formula vi) L-(X′⁻)ₓ with x=2 and X′⁻=—OSO₃⁻ or —SO₃⁻ as defined previously, and more particularly L represents a linear or branched C₄-C₄₀ alkylene group, optionally substituted with at least one hydroxyl group, preferably located beta to the sulfonate groups; the said alkylene being optionally interrupted with one or more oxygen atoms —O— and/or arylene such as phenylene.

More particularly, X⁻ is the sulfate/sulfonate 71 below:

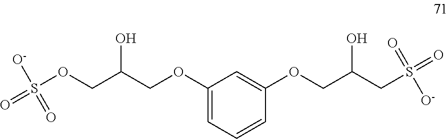 [SMILES: O=S(=O)([O-])CC(O)COc1cccc(OCC(O)COS(=O)(=O)[O-])c1]

According to a preferred embodiment of the invention, X⁻ represents a monophosphate belonging to formula iv) R_{d}—O—PO₃²⁻ as defined previously, and preferably with R_{d} representing a linear or branched C₂-C₄₀ alkyl or linear or branched C₂-C₄₀ alkenyl group comprising from 1 to 2 unsaturations, the alkyl or alkenyl group being optionally substituted with at least one hydroxyl or (di)(C₁-C₄)(alkyl)amino group; or alternatively R_{d} represents a (hetero)cycloalkyl(C₁-C₄)alkyl group optionally substituted with one or more hydroxyl groups. Preferentially, the radical R_{c} represents a 5- or 6-membered heterocycloalkyl such as pyranose or furanose. More particularly, X⁻ is chosen from the monophosphates 72 to 83 below, and also optical or geometrical isomers thereof:

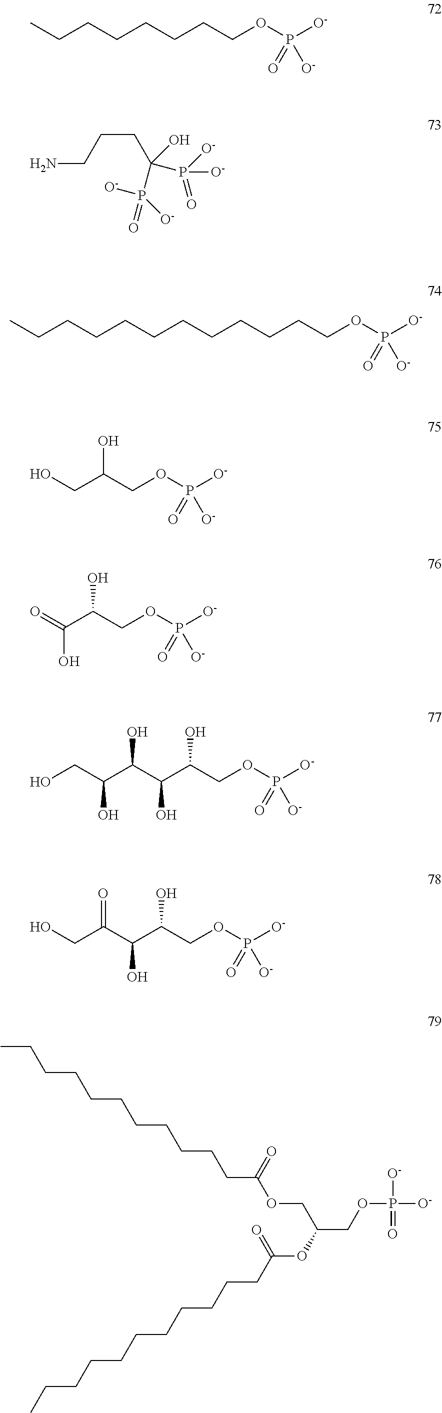

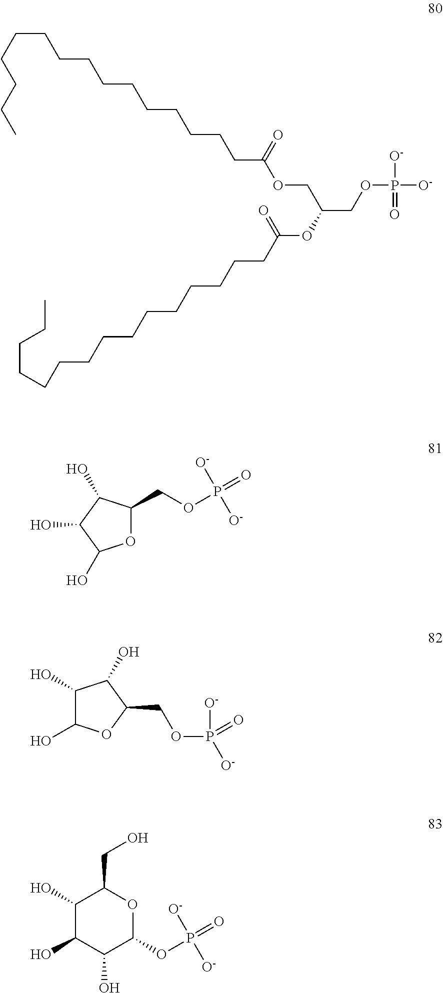 [SMILES: CCCCCCCCCCCCCCCC(=O)OC[C@H](COP(=O)([O-])[O-])OC(=O)CCCCCCCCCCCCCCC ; O=P([O-])([O-])OC[C@H]1OC(O)[C@H](O)[C@@H]1O ; O=P([O-])([O-])OC[C@H]1OC(O)[C@H](O)[C@@H]1O ; O=P([O-])([O-])O[C@H]1O[C@H](CO)[C@@H](O)[C@H](O)[C@H]1O]

According to a preferred embodiment of the invention, X⁻ represents a carboxylate/phosphate mixed counterion belonging to the formula vi) L-(X′⁻)ₓ with x=2 and X′⁻═CO₂⁻ or O—PO₃²⁻ as defined previously, and more particularly L represents a linear or branched C₄-C₄₀ alkylene group, optionally substituted with at least one hydroxyl group, preferably located alpha to the carboxylate group or beta to the phosphate group.

More particularly, X⁻ is the carboxylate/phosphate 84 below, and also optical or geometrical isomers thereof:

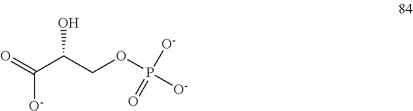 [SMILES: O=C([O-])[C@H](O)COP(=O)([O-])[O-]]

According to a preferred embodiment of the invention, X⁻ represents an anionic oligomer or polymer comprising at least one group X′⁻ as defined previously. The following are envisaged as examples of anionic oligomers or polymers:
Polycarboxylates, polysulfates or polysulfonates derived, for example, from alginic acid or hyaluronic acid, polymethacrylic acids, lignosulfonic acid, polylactic acid, acrylic acid polymers, etc.
According to a preferred embodiment of the invention, X⁻ represents viii) an anionic oligomer or polymer comprising as anionic group sulfonate or carboxylate, and more preferably anionic polymer derived from polysaccharide with sulfonate or carboxylate groups such alginate, carboxymethylcellulose.

Very particularly the counter anion X⁻ of the invention is chosen from the species i), ii), iii) and viii), more particularly X⁻ is chosen from i), ii) and iii), and preferably X⁻ represents a monocarboxylate belonging to formula i) Rₐ—CO₂⁻ as defined herein before.

The Cationic Part Col⁺

The cationic direct dyes of formula (I) according to the invention are “derived” from dyes commonly known as “basic dyes” for their affinity with acidic substances (see, for example, “Industrial Dyes, Chemistry, Properties, Application”, Klaus Hunger Ed. Wiley-VCH Verlag GmbH & Co KGaA, Weinheim 2003). Basic or cationic dyes are known in the literature (see, for example, “Ullman's Encyclopedia of Industrial Chemistry”, 2012 Wiley-VCH Verlag GmbH & Co. KGaA, Weinheim doi/10.1002/14356007.a26_{—}351, point 4.8.4; Cationic Dyestuffs, Review of Progress in Coloration and Related Topics, Volume 14, Issue 1, June 1984, Pages: 187-203).

The term “cationic direct dyes” means any direct dye comprising in its structure at least one intrinsic (endocyclic) cationic group and/or exogenous (exocyclic) cationic group of formula (1), (2) or (2′).

As cationic part derived from cationic dyes that are useful in the present invention, mention may be made of those derived from the following cationic dyes: acridines; acridones; anthranthrones; anthrapyrimidines; anthraquinones; azines; (poly)azos, hydrazono or hydrazones, in particular arylhydrazones; azomethines; benzanthrones; benzimidazoles; benzimidazolones; benzindoles; benzoxazoles; benzopyrans; benzothiazoles; benzoquinones; bisazines; bis-isoindolines; carboxanilides; coumarins; cyanins such as azacarbocyanins, diazacarbocyanins, diazahemicyanins, hemicyanins, or tetraazacarbocyanins; diazines; diketopyrrolopyrroles; dioxazines; diphenylamines; diphenylmethanes; dithiazines; flavonoids such as flavanthrones and flavones; fluorindines; formazans; indamines; indanthrones; indigoids and pseudo-indigoids; indophenols; indoanilines; isoindolines; isoindolinones; isoviolanthrones; lactones; (poly)methines such as dimethines of the type such as stilbene or styryl; naphthalimides; naphthanilides; naphtholactams; naphthoquinones; nitro, especially nitro(hetero)aromatics; oxadiazoles; oxazines; perilones; perinones; perylenes; phenazines; phenoxazine; phenothiazines; phthalocyanin; porphyrins; pyranthrones; pyrazolanthrones; pyrazolones; pyrimidinoanthrones; pyronines; quinacridones; quinolines; quinophthalones; squaranes; tetrazoliums; thiazines; triarylmethanes, or xanthenes.

Among the azo compounds, mention may be made more particularly of those in the Kirk Othmer Encyclopedia of Chemical Technology, “Dyes, Azo”, J. Wiley & Sons, updated on Apr. 19, 2010.

In particular, the cationic part derived from a cationic dye of formula (I) is chosen from those derived from the following cationic dyes: nitro dyes, anthraquinone dyes, (poly)azo dyes such as (di)azo dyes, hydrazono dyes, (poly)methine dyes such as styryl dyes, tetraazapentamethine dyes, phenoxazine dyes, phenazine dyes, phenothiazine dyes, anthraquinone dyes, naphthalimide dyes, triarylmethane dyes or phthalocyanin dyes, each of these dyes having at least one endocyclic or exocyclic cationic group of formulae (1), (2) and (2′) as defined previously, bearing an anionic counterion X⁻ as defined previously.

According to a preferred embodiment of the invention, in formula (I) of the invention, the radical Col⁽⁺⁾m represents the cationic part of the “basic dyes” or of the cationic direct dyes that are “derived from” the said basic dyes or from the said cationic direct dyes. The dyes of the invention derived from basic direct dyes or basic dyes or from the said cationic direct dyes thus comprise at least one anionic counterion X⁻ as defined previously.

Mention may be made of the hydrazono cationic part of formulae (IIIa) and (III′a), the azo cationic parts (IVa) and (IV′a) and the diazo cationic parts (Va) below:

| | | |
|---|---|---|
| Het⁺—C(R^{a})═N—N(R^{b})—Ar | Het⁺—N(R^{a} )—N═C(R^{b})—Ar | Het⁺—N═N—Ar |
| (IIIa) | (III′a) | (IVa) |
| Ar⁺—N═N—Ar″ | and | Het⁺—N═N—Ar′—N═N—Ar |
| (IV′a) | | (Va) |

in which formulae (IIIa), (III′a), (IVa), (IV′a) and (Va):
Het⁺ represents a cationic heteroaryl radical, preferably bearing an endocyclic cationic charge, such as imidazolium, indolium or pyridinium, optionally substituted preferentially with one or more (C₁-C₈) alkyl groups such as methyl;
Ar⁺ representing an aryl radical, such as phenyl or naphthyl, bearing an exocyclic cationic charge, preferentially ammonium, particularly tri(C₁-C₈)alkylammonium such as trimethylammonium;
Ar⁺ represents an aryl group, especially phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar′ is an optionally substituted divalent (hetero)arylene group such as phenylene, particularly para-phenylene, or naphthalene, which are optionally substituted, preferentially with one or more groups (C₁-C₈)alkyl, hydroxyl or (C₁-C₈)alkoxy;
Ar″ is an optionally substituted (hetero)aryl group such as phenyl or pyrazolyl, which are optionally substituted, preferentially with one or more groups (C₁-C₈)alkyl, hydroxyl, (di)(C₁-C₈)(alkyl)amino, (C₁-C₈)alkoxy or phenyl;
R^{a} and R^{b}, which may be identical or different, represent a hydrogen atom or a group (C₁-C₈)alkyl, which is optionally substituted, preferentially with a hydroxyl group;
or alternatively the substituent R^{a} with a substituent of Het⁺ and/or R^{b} with a substituent of Ar and/or R^{a} with R^{b} form, together with the atoms that bear them, a (hetero)cycloalkyl;
particularly, R^{a} and R^{b} represent a hydrogen atom or a group (C₁-C₄)alkyl, which is optionally substituted with a hydroxyl group.

In particular, mention may be made of the azo and hydrazono cationic parts bearing an endocyclic cationic charge of formulae (IIIa), (III′a) and (IVa) as defined previously. More particularly those of formulae (IIIa), (III′a) and (IVa) derived from the dyes described in patent applications WO 95/15144, WO 95/01772 and EP-714954.

Preferentially, the cationic part is derived from the following derivatives:

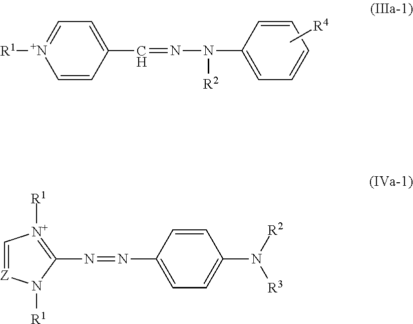

formulae (III-1) and (IV-1) with:
R¹ representing a (C₁-C₄) alkyl group such as methyl;
R² and R³, which are identical or different, represent a hydrogen atom or a (C₁-C₄)alkyl group, such as methyl; and
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; particularly, R⁴ is a hydrogen atom,
Z represents a CH group or a nitrogen atom, preferentially CH.

Particularly, the cationic part of formulae (IIIa-1) and (IVa-1) is chosen from Basic Red 51, Basic Yellow 87 and Basic Orange 31 or derivatives thereof:

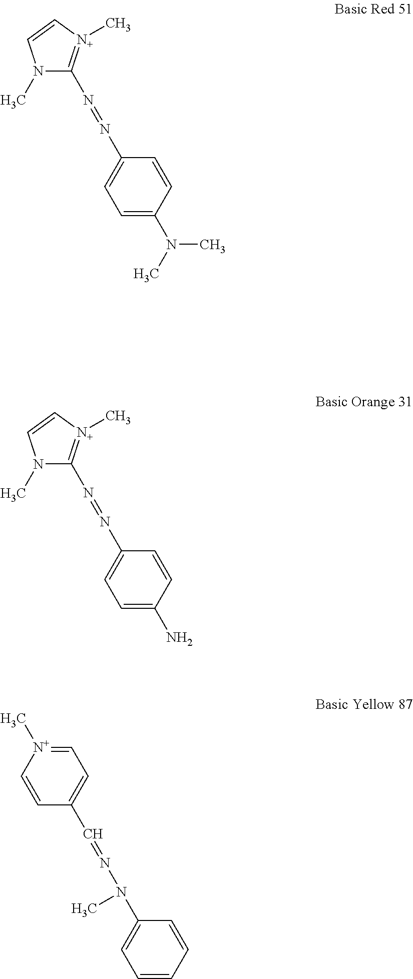

According to a particular embodiment of the invention, the dyes of formula (I) are fluorescent, i.e. they contain a fluorescent cationic part Col⁺.

As cationic parts of the present invention, mention may be made of those derived from the following cationic dyes: acridines, acridones, benzanthrones, benzimidazoles, benzimidazolones, benzindoles, benzoxazoles, benzopyrans, benzothiazoles, coumarins, difluoro{2-[(2H-pyrrol-2-ylidene-kN)methyl]-1H-pyrrolato-kN}bores (BODIPY®), diketopyrrolopyrroles, fluorindines, (poly)methines (especially cyanins and styryls/hemicyanins), naphthalimides, naphthanilides, naphthylamine (such as dansyls), oxadiazoles, oxazines, perilones, perinones, perylenes, polyenes/carotenoids, squaranes, stilbenes and xanthenes.

Mention may also be made of the fluorescent cationic part derived from cationic dyes described in documents EP 1 133 975, WO 03/029 359, EP 860 636, WO 95/01772, WO 95/15144, EP 714 954 and those listed in the encyclopaedia The chemistry of synthetic dyes by K. Venkataraman, 1952, Academic Press, vol. 1 to 7, in Kirk Othmer's encyclopaedia Chemical Technology, in the chapter “Dyes and dye Intermediates”, 1993, Wiley and Sons, and in various chapters of Ullmann's Encyclopedia of Industrial Chemistry 7th edition, Wiley and Sons, and in The Handbook—A Guide to Fluorescent Probes and Labeling Technologies, 10th Ed Molecular Probes/Invitrogen-Oregon 2005 circulated on the Internet or in the preceding printed editions.

According to a preferred embodiment of the invention, when the cationic dye of the invention is derived from an azo dye, in particular of formula (IVa), then its anionic counterion X⁻ is other than i) carboxylates. Preferably, when the dye according to the invention is derived from an azo dye, the associated anionic counterion is chosen from sulfonates ii) R_{b}—SO₃⁻; sulfates iii) R_{c}—O—SO₃⁻; phosphates iv) R_{d}—O—P(O)(OH)O⁻ or R_{d}—O—PO₃²⁻; phosphate esters v) Rₑ—P(OR_{f})O₂⁻; the following polyanionic counterions vi) L-(X′)ₓ as defined previously.

According to a preferred variant of the invention, the cationic part of the compounds of formula (I) according to the invention is derived from the polymethine dyes of formulae (VIa) and (VI′a) below:

W⁺—[C(R^{c})═C(R^{d})]_{m′}—Ar′ (VIa)

Ar—[C(R^{d})═C(R^{c})]_{m′}—W′⁺ (VI′a)

formula (VIa) or (VI′a) with:
W⁺ representing a cationic heterocyclic or heteroaryl group, particularly comprising a quaternary ammonium optionally substituted with one or more (C₁-C₈)alkyl groups optionally substituted especially with one or more hydroxyl groups;
W′⁺ representing a heterocyclic or heteroaryl radical as defined for W⁺;
Ar representing a (hetero)aryl group such as phenyl or naphthyl, optionally substituted preferentially with i) one or more halogen atoms such as chlorine or fluorine; ii) one or more groups (C₁-C₈)alkyl, preferably of C₁-C₄ such as methyl; iii) one or more hydroxyl groups; iv) one or more (C₁-C₈)alkoxy groups such as methoxy; v) one or more hydroxy(C₁-C₈)alkyl groups such as hydroxyethyl, vi) one or more amino groups or (di)(C₁-C₈)alkylamino, preferably with the C₁-C₄ alkyl part optionally substituted with one or more hydroxyl groups, such as (di)hydroxyethylamino, vii) with one or more acylamino groups; viii) one or more heterocycloalkyl groups such as piperazinyl, piperidyl or 5- or 6-membered heteroaryl such as pyrrolidinyl, pyridyl and imidazolinyl;
Ar′ is a (hetero)aryl radical as defined for Ar;
m′ represents an integer between 1 and 4 inclusive, and in particular m is 1 or 2; more preferentially 1;
R^{c} and R^{d}, which may be identical or different, represent a hydrogen atom or an optionally substituted (C₁-C₈)alkyl group, preferentially of C₁-C₄, or alternatively RC contiguous with W⁺ or W′⁺ and/or R^{d} contiguous with Ar or Ar′ and/or contiguous R^{c} and R^{d} form, with the atoms that bear them, a (hetero)cycloalkyl, particularly R^{c} is contiguous with W⁺ or W′⁻ and forms a (hetero)cycloalkyl such as cyclohexyl.

According to another variant, the cationic part is derived from a quaternized fluorescent dye such that, in formula (I), Col⁺ represents a naphthalimide radical bearing an exocyclic cationic charge of formula (VIIa):

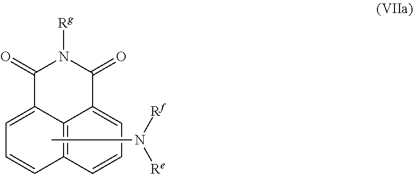

in which formula (VIIa) R^{e}, R^{f}, R^{g} and R^{h}, which may be identical or different, represent a hydrogen atom or a C₁-C₆ alkyl group which is optionally substituted, preferentially with a di(C₁-C₆)alkylamino or tri(C₁-C₆)alkylammonium group such as trimethylammonium.

Preferably, W⁺ or W′⁺ is an imidazolium, pyridinium, benzimidazolium, pyrazolium, benzothiazolium or quinolinium optionally substituted with one or more identical or different C₁-C₄ alkyl radicals.

According to a particularly preferred embodiment of the invention, the cationic part is derived from a dye of formula (VI′a) as defined previously with m′=1, Ar representing a phenyl group substituted para to the styryl group —C(R^{d})═C(R^{c})— with a group (di)(hydroxy)(C₁-C₆)(alkyl)amino such as dihydroxy(C₁-C₄)alkylamino, and W′⁺ representing an imidazolium or pyridinium group, preferentially ortho- or para-pyridinium.

According to another preferred embodiment of the invention, Col⁽⁺⁾X⁻ comprises in its structure:
at least one heteroaryl bearing an endocyclic cationic charge, i.e. intrinsic to the structure of the dye; and/or
at least one (hetero)aryl or (hetero)cycloalkyl group bearing an exocyclic cationic charge;
   the cationic charge being of formula (1), (2), (2′) as defined previously.

According to a particular embodiment of the invention, the dyes of formula (I) are such that m is equal to n.

One variant of the invention concerns the dyes of formula (I) for which m and n represent an integer between 1 and 10 and more particularly between 1 and 4, such as between 1 and 3.

The preferred cationic dyes of formula (I) of the invention are chosen from basic nitro direct dyes, basic azo dyes, basic anthraquinone dyes, basic triarylmethane dyes, basic hydrazono dyes, basic tetraazapentamethine dyes, anionic styryl dyes, basic phenoxazine dyes, basic phenothiazine dyes, basic phenazine dyes, basic phthalocyanin dyes and basic natural dyes; each of these dyes having at least one endocyclic or exocyclic cationic group of formulae (1), (2) and (2′) bearing an anionic counterion X⁻ as defined previously.

As cationic dyes of formula (I) according to the invention, mention may be made particularly of the following dyes of formulae (II), (II′), (III), (IV), (V), (V′), (VI), (VII), (VIII), (VIII′), (VIII″), (VIII′″) (IX), (X), (XI), (XII) and (XIII) bearing an endocyclic or internal cationic charge and/or bearing an exocyclic or external cationic charge:

a) the cationic azo dyes of formula (II) or (II′):

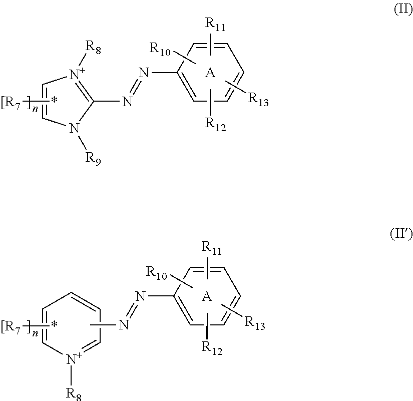

in which formulae (II) and (II′):
R₈, and R₉, which may be identical or different, represent a C₁-C₈ alkyl radical optionally substituted with at least one hydroxyl group or a tri(C₁-C₆)alkylsilane group;
R₇ represents a hydrogen atom, a C₁-C₆ alkyl radical, or alternatively two radicals R₇ located ortho to each other when n is greater than or equal to 2 together form an aryl group such as benzo optionally substituted with a hydroxyl, (C₁-C₆)alkoxy, amino or (di)(C₁-C₆)(alkyl)amino group;
n is an integer equal to 1, 2 or 3;
R₁₀, R₁₁, R₁₂ and R₁₃, which may be identical or different, represent a hydrogen atom or a group chosen from:
   C₁-C₆ alkyl;
   alkoxy, alkylthio;
   hydroxyl;
   amino;
   (di)(C₁-C₆)alkylamino, the alkyl group possibly being substituted with a hydroxyl group;
   R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
   R″—S(O)₂—, with R″ representing a hydrogen atom or an alkyl, aryl, (di)(alkyl)amino or aryl(alkyl)amino group; preferentially a phenylamino or phenyl group;
   R′″—S(O)₂—X′— with R′″ representing an alkyl or optionally substituted aryl group, X′ as defined previously;
   aryl(alkyl)amino optionally substituted with one or more groups chosen from i) amino; ii) alkoxy;
   Ar—N═N— with Ar representing an optionally substituted aryl group;
   or alternatively two contiguous groups R₁₀ with R₁₁ or R₁₁ with R₁₂ or R₁₂ with R₁₃ together form a fused benzo group A′; with A′ optionally substituted with one or more groups chosen from i) amino; ii) (di)(C₁-C₆ alkyl)amino; iii) alkoxy; iv) hydroxyl; v) R^{o}—C(X)—X′—; vi) R^{o}—X′—C(X)—; vii) R^{o}—X′—C(X)—X″—; and viii) optionally substituted aryl(alkyl)amino; with X⁺, R^{o}, X, X′ and X″ as defined previously.

As examples of dyes of formulae (II) and (II′), mention may be made of salts derived from Basic Red 51 and Basic Orange 31, and those present in patent applications FR0104537, FR0104466, FR0104467, FR0104468, FR0112374 and FR0211186.

According to a preferred embodiment of the invention, when the cationic dye of the invention is derived from an azo dye, in particular of formula (II) or (II′), then its anionic counterion X⁻ is other than i) carboxylates. Preferably, when the dye according to the invention is derived from an azo dye, the associated anionic counterion is chosen from sulfonates ii) R_{b}—SO₃⁻; sulfates iii) R_{c}—O—SO₃⁻; phosphates iv) R_{d}—O—P(O)(OH)O⁻ or R_{d}—O—PO₃₂; phosphate esters v) Rₑ—P(OR_{f})O₂⁻; the following polyanionic counterions vi) L-(X′)ₓ as defined previously; or viii) as defined herein before

b) the pyrazolone cationic azo dyes of formula (III):

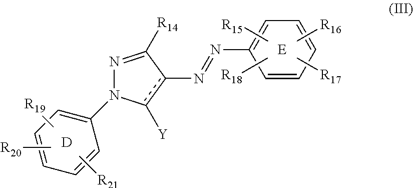

in which formula (III):
R₁₉, R₂₀ and R₂₁, which may be identical or different, represent a hydrogen or halogen atom, an alkyl group or a cationic group of formulae (1), (2) and/or (2′) as defined previously;
R₁₄ represents a hydrogen atom or a C₁-C₆ alkyl group;
R₁₅, R₁₆, R₁₇ and R₁₈, which may be identical or different, represent a hydrogen atom or a group chosen from:
   a cationic group of formulae (1), (2) and/or (2′) as defined previously;
   a hydroxyl;
   a C₁-C₄ alkoxy;
   an amino;
   a (di)(C₁-C₆ alkyl)amino;
R₁₅ and R₁₆ or R₁₆ and R₁₇ or R₁₇ and R₁₈ may together form an optionally substituted benzo group E′;
Y represents either a hydroxyl group or an oxo group;
[symbol] represents a single bond when Y is an oxo group; and represents a it being understood that formula (III) comprises at least one cationic group of formulae (1), (2) and/or (2′) as defined previously on one of the rings D or E.

As examples of dyes of formula (III), mention may be made of salts derived from: Basic Yellow 57;

c) the anthraquinone dyes of formula (IV):

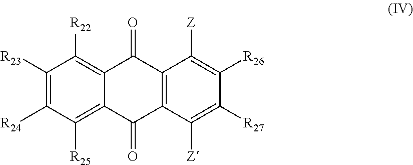

in which formula (IV):
R₂₂, R₂₃, R₂₄, R₂₅, R₂₆ and R₂₇, which may be identical or different, represent a hydrogen or halogen atom or a group chosen from:
   alkyl;
   hydroxyl, mercapto;
   alkoxy, alkylthio;
   optionally substituted aryloxy or arylthio, preferentially substituted with one or more groups chosen from alkyl and a cationic group of formulae (1), (2) and/or (2′) as defined previously;
   aryl(alkyl)amino optionally substituted with one or more groups chosen from alkyl and a cationic group of formulae (1), (2) and/or (2′) as defined previously;
   (di)(alkyl)amino;
   (di)(hydroxyalkyl)amino;
   a cationic group of formulae (1), (2) and/or (2′) as defined previously;
Z′ represents a hydrogen atom or an NR₂₈R₂₉ group with R₂₈ and R₂₉, which may be identical or different, representing a hydrogen atom or a group chosen from:
   alkyl;
   polyhydroxyalkyl such as hydroxyethyl;
   aryl optionally substituted with one or more groups, particularly i) alkyl such as methyl, n-dodecyl, n-butyl; ii) a cationic group of formulae (1), (2) and/or (2′) as defined previously; iii) R^{o}—C(X)—X′—, R^{o}—X′—C(X)—, R^{o}—X′—C(X)—X″— with R^{o}, X, X′ and X″ as defined previously, preferentially R^{o} represents an alkyl group;
   cycloalkyl; especially cyclohexyl;
Z, represents a group chosen from hydroxyl and NR′₂₈R′₂₉ with R′₂₈ and R′₂₉, which may be identical or different, representing the same atoms or groups as R₂₈ and R₂₉ as defined previously;
   it being understood that formula (IV) comprises at least one cationic group of formulae (1), (2) and/or (2′) as defined previously.

As examples of dyes of formula (IV), mention may be made of the salts mentioned, for example, in patent US005891200.

d) the hydrazone dyes of formulae (V) and (V′):

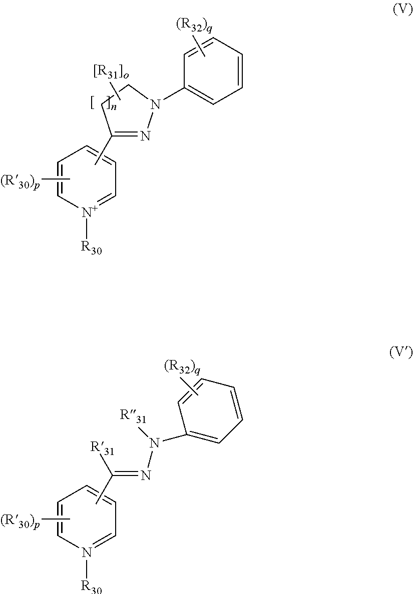

in which formulae (V) and (V′):
n is an integer equal to 1 or 2;
o is an integer between 0 and 4 inclusive;
p is an integer between 0 and 4 inclusive;
q is an integer between 0 and 5 inclusive;
R₃₀, independently of each other, represent:
   an optionally substituted C₁-C₂₀ alkyl radical, optionally interrupted with one or more heteroatoms and/or with one or more groups comprising at least one heteroatom, preferably chosen from oxygen, sulfur, C(O), S(O), S(O)₂ or combinations thereof or with a cationic group of formulae (1), (2) and/or (2′) as defined previously;
   a C₁-C₄ trialkylsilyl radical;
   an optionally substituted phenyl radical;
   an optionally substituted benzyl radical;
R′₃₀, independently of each other, represent:
   a halogen atom;
   an optionally substituted C₁-C₁₆ alkyl radical;
   a hydroxyl radical;
   a C₁-C₄ alkoxy radical;
   an amino radical optionally substituted with one or two identical or different C₁-C₄ alkyl radicals, optionally bearing at least one hydroxyl group;
   an alkylcarbonylamino radical (R—C(O)—N(R′)—) in which the radical R represents a C₁-C₄ alkyl radical and R′ represents a hydrogen atom or a C₁-C₄ alkyl radical;
   an alkylsulfonylamino radical (R—S(O)₂—N(R′)—) in which the radical R represents a C₁-C₄ alkyl radical and the radical R′ represents a hydrogen atom or a C₁-C₄ alkyl radical;
   or alternatively two radicals R′₃₀, which are adjacent when p is greater than or equal to 2, form, together with the carbon atoms to which they are attached, a substituted or unsubstituted, 5- or 6-membered aromatic or non-aromatic (hetero)cyclic radical;
R₃₁, independently of each other, represent a hydrogen atom, an optionally substituted C₁-C₁₆ alkyl radical or an optionally substituted (hetero)aryl radical, or alternatively two adjacent radicals R₃₁, when o is greater than or equal to 2, form, with the carbon atoms that bear them, an optionally substituted, optionally unsaturated, 5- to 7-membered (hetero)cycle, optionally fused to another aromatic nucleus, optionally comprising another nitrogen or non-nitrogen heteroatom;
R′₃₁ represents a hydrogen atom or an optionally substituted C₁-C₁₆ alkyl radical;
R″₃₁ represents a hydrogen atom or a radical chosen from optionally substituted C₁-C₂₀ alkyl; optionally substituted phenyl; optionally substituted benzyl; alkylcarbonyl (R—C(O)—) in which R represents a C₁-C₄ alkyl radical; alkylsulfonyl (R—S(O)₂—) in which R represents a C₁-C₄ alkyl radical; arylsulfonyl (R′—S(O)₂—) in which R′ represents an optionally substituted phenyl or benzyl radical;
R₃₂ represents a hydrogen atom, a halogen atom such as bromine, chlorine or fluorine, or a radical chosen from optionally substituted C₁-C₂₀ alkyl; hydroxyl; C₁-C₄ alkoxy; alkylthio (R—S—) in which the group R represents a C₁-C₄ alkyl radical; alkoxycarbonyl (R—O—C(O)—) in which R is as defined previously; alkylcarbonyloxy (R—C(O)—O—) in which R is as defined previously; optionally substituted aryloxy; alkylcarbonylamino (R—C(O)—N(R′)—) in which the radical R is as defined previously, and R′ represents a hydrogen atom or a C₁-C₄ alkyl radical; ureido ((R)₂N—CO—NR′—) in which the radicals R and R′, which may be identical or different, are as defined previously; and amino —NR′₃₃R″₃₃ in which R′₃₃ and R″₃₃, which may be identical or different, represent:
   a hydrogen atom;
   a (C₁-C₄)alkyl radical optionally substituted with at least one hydroxyl or C₁-C₂ alkoxy group, the said alkyl radicals possibly forming, with the nitrogen atom that bears them, an optionally aromatic, optionally substituted, saturated or unsaturated, 5- or 7-membered heterocycle, optionally comprising another nitrogen or non-nitrogen heteroatom;
   a phenyl radical;
   an aminophenyl radical;
   a 4-N,N-diethylaminophenyl radical; or
   a methoxyphenyl radical;
or alternatively when q is greater than or equal to 2, two adjacent radicals R₃₂ form, with the carbon atoms that bear them, a substituted or unsubstituted, 5- or 6-membered aromatic or non-aromatic (hetero)cyclic radical;
or alternatively R″₃₁ and R₃₂ located ortho to the NR″₃₁ group form, with the nitrogen atom substituted with R″₃₁, a saturated or unsaturated, substituted or unsubstituted 5- or 6-membered heterocycle;
and/or R′₃₁ and R′₃₀ located ortho to the hydrazono group form, with the carbon atoms that bear them, a saturated or unsaturated, substituted or unsubstituted 5- or 6-membered heterocycle.

According to an advantageous variant of the invention, the compounds of formula (V′) are such that one of the radicals R′₃₃ or R″₃₃ forms, with the nitrogen atom that bears it and with a radical R₃₂ located ortho to the NR′₃₃R″₃₃ group, a saturated or unsaturated, substituted or unsubstituted 5- or 6-membered heterocycle; such as the following groups:

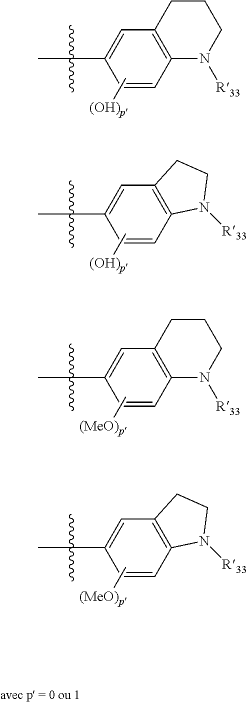

As examples of dyes of formulae (V) and (V′), mention may be made of salts derived from Basic Yellow 87 and those derived from patent applications FR0603322, FR0754454 and FR0858801.

e) the triarylmethane dyes of formula (VI):

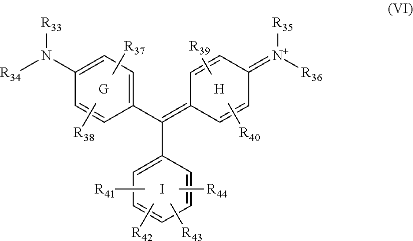

in which formula (VI):
R₃₃, R₃₄, R₃₅ and R₃₆, which may be identical or different, represent a hydrogen atom or a group chosen from (C₁-C₆)alkyl, optionally substituted aryl and optionally substituted aryl(C₁-C₆)alkyl; particularly a (C₁-C₄)alkyl or benzyl group optionally substituted with a cationic group of formulae (1), (2) and/or (2′) as defined previously;
R₃₇, R₃₈, R₃₉, R₄₀, R₄₁, R₄₂, R₄₃ and R₄₄, which may be identical or different, represent a hydrogen atom or a group chosen from:
   (C₁-C₄)alkyl;
   (C₁-C₄)alkoxy, (C₁-C₄)alkylthio;
   (di)(C₁-C₄)(alkyl)amino;
   hydroxyl, mercapto;
   R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or a (C₁-C₄)alkyl group;
   or alternatively two contiguous groups R₄₁ with R₄₂ or R₄₂ with R₄₃ or R₄₃ with R₄₄ together form a fused benzo group: I′; with I′optionally substituted with one or more groups chosen from i) hydroxyl; ii) mercapto; iii) (di)(C₁-C₄)(alkyl)amino; iv) R^{o}—C(X)—X′—; v) R^{o}—X′—C(X)—; vi) R^{o}—X′—C(X)—X″—; with R^{o}, X, X′, X″ as defined previously;
      in particular, R₃₇ to R₄₀ represent a hydrogen atom, and R₄₁ to R₄₄, which may be identical or different, represent a hydroxyl group or cationic group of formulae (1), (2) and/or (2′) as defined previously; and when R₄₃ with R₄₄ together form a benzo group, it is preferentially substituted with a cationic group of formulae (1), (2) and/or (2′) as defined previously.

As examples of dyes of formula (VI), mention may be made of salts derived from Basic Green 4, Basic Violet 1, Basic Violet 3, Basic Violet 4, Basic Blue 5, Basic Blue 7, Basic Blue 11, Basic Blue 26, Basic Violet 14, Basic Blue 20, Basic Green 1, Brilliant Basic Cyanine.

f) the xanthene-based dyes of formula (VII):

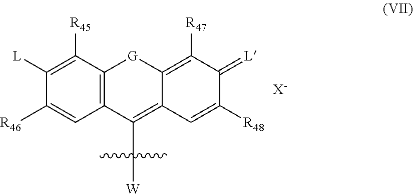

in which formula (VII):
W represents a hydrogen or the radical below:

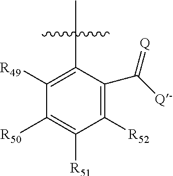

R₄₅, R₄₆, R₄₇ and R₄₈, which may be identical or different, represent a hydrogen atom or a (C₁-C₄)alkyl and particularly C₁-C₂ alkyl radical, or a halogen atom;
R₄₉, R₅₀, R₅₁ and R₅₂, which may be identical or different, represent a hydrogen or halogen atom or a group chosen from:
   (C₁-C₄)alkyl;
   (C₁-C₄)alkoxy, (C₁-C₄)alkylthio;
   hydroxyl, mercapto, amino; particularly R₅₃ R₅₄, R₅₅ and R₄₈ represent a hydrogen or halogen atom, a methyl radical or an amino radical;
G and Q, which may be identical or different, represent an oxygen or sulfur atom or a group NRₑ with Re as defined previously; particularly G represents an oxygen atom and/or Q represents an oxygen atom;
L represents a group NR_{f}R_{g}, with R_{f} and R_{g} representing, independently of each other, a hydrogen atom or a (C₁-C₄)alkyl or optionally substituted aryl group;
L′ represents an ammonium group: N+R_{f}R_{g}, with R_{f} and R_{g}, as defined previously;
Q′ represents a hydroxyl or C₁-C₆ alkoxy radical;
X⁻ is as defined previously.

As examples of dyes of formula (VII), mention may be made of salts derived from: Basic Red 1, Basic Violet 10, Pyronine Yellow (CI 45005), Pyronine B (CI 45010), Tetramethylrhodamine, Rhodamine 3B, Rhodamine 19, Acridine Red 3B, Rhodamine 116, Rhodamine B Amine, Rhodamine B Hexyl Ester.

f) the styryl dyes of formulae (VIII), (VIII′), (VIII″), (VIII′″):

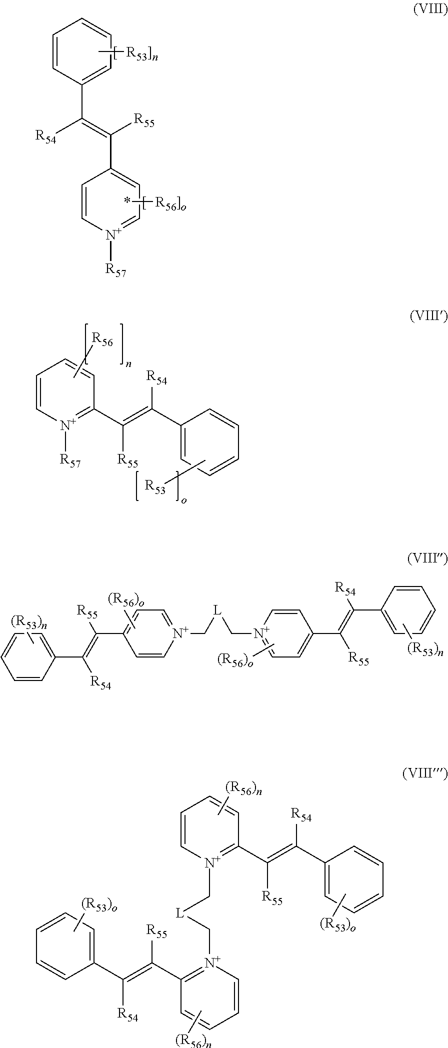

in which formulae (VIII), (VIII′), (VIII″) and (VIII′):
R₅₃, which may be identical or different, represent:
   a hydrogen atom;
   a halogen atom;
   a linear or branched (C₁-C₆)alkyl radical comprising 1 to 4 carbon atoms, optionally substituted and/or interrupted with at least one heteroatom and/or group bearing at least one heteroatom and/or substituted with at least one halogen atom;
   a hydroxyl radical;
   a (C₁-C₆)alkoxy radical;
   a radical NRᵢRⱼ in which Rᵢ and Rⱼ, which may be identical or different, represent:
      a hydrogen atom;
      a C₁-C₄ alkyl radical optionally bearing at least one hydroxyl or C₁-C₂ alkoxy group, the said alkyl radicals possibly forming, with the nitrogen atom to which they are attached, an optionally aromatic, optionally substituted, saturated or unsaturated, 5- or 7-membered heterocycle, optionally comprising another nitrogen or non-nitrogen heteroatom;
      an aryl radical such as phenyl;
   an alkylcarbonylamino group (R—C(O)—N(R′)—) in which the radical R represents a C₁-C₄ alkyl radical and the radical R′ represents a hydrogen atom or a C₁-C₄ alkyl radical;
   a ureido group ((R)₂N—C(O)—N(R′)—) in which the radicals R and R′, which may be identical or different, represent a hydrogen atom or a C₁-C₄ alkyl radical;
n is an integer between 1 and 5;
R₅₄ and R₅₅, which may be identical or different, represent a hydrogen atom or a C₁-C₄ alkyl radical;
R₅₆, which may be identical or different, represent a hydrogen atom, a halogen atom, a linear or branched alkyl radical comprising 1 to 4 carbon atoms, optionally interrupted with at least one heteroatom, a hydroxyl;
o is an integer between 1 and 4;
R₅₇, which may be identical or different, represent a radical chosen from:
   optionally substituted C₁-C₂₀ alkyl, optionally interrupted with one or more heteroatoms and/or with one or more groups comprising at least one heteroatom, preferably chosen from oxygen, sulfur, C(O), S(O), S(O)₂ or combinations thereof or a cationic group of formulae (1), (2) and/or (2′) as defined previously;
   C₁-C₄ trialkylsilyl;
   optionally substituted phenyl; and
   optionally substituted benzyl;
L represents a divalent linker arm chosen from:
   a linear or branched alkylene radical comprising 1 to 14 carbon atoms or an alkenylene radical comprising 2 to 14 carbon atoms, optionally substituted and/or interrupted with at least one heteroatom chosen from oxygen, sulfur, nitrogen or divalent groups —N(R)—, —C(O)—, —C(S)— or a combination thereof, with R representing a hydrogen atom or a (C₁-C₆)alkyl group, the said alkylene radical possibly being interrupted also with a (hetero)arylene group, or divalent (hetero)cycle;
   a 5- or 6-membered divalent (hetero)cyclic radical, optionally substituted with at least one linear or branched alkyl radical comprising 1 to 14 carbon atoms, optionally substituted with at least one heteroatom; with at least one amino(C₁-C₆)alkyl radical optionally substituted with at least one heteroatom; with at least one halogen atom;
   a fused or non-fused (hetero)arylene radical, optionally separated with an alkyl radical comprising 1 to 4 carbon atoms, the aryl radical(s) being optionally substituted with at least one halogen atom or with at least one alkyl radical comprising 1 to 10 carbon atoms optionally substituted and/or interrupted with at least one heteroatom and/or group bearing at least one heteroatom;
      it being understood that the linker arm L may bear one or more cationic groups of formulae (1), (2) and/or (2′) as defined previously.
      g) the naphthalimide dyes of formula (IX):

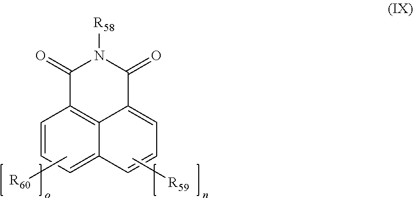

in which formula (IX):
R₅₈ represents a hydrogen atom, an aryl or aralkyl radical in which the aryl part is optionally substituted; an optionally substituted C₁-C₈ and preferably C₂-C₈ alkyl radical, optionally interrupted with one or more heteroatoms chosen from oxygen, nitrogen and sulfur and possibly being substituted with at least one cationic group of formulae (1), (2) and/or (2′) as defined previously:
R₅₉ and R₆₀, which may be identical or different, represent i) a hydrogen atom; or a radical chosen from ii) —SRₖ with Rₖ representing a (C₁-C₆)alkyl radical optionally substituted with a hydroxyl; iii) —ORₗ with Rₗ representing a (C₁-C₆)alkyl radical optionally substituted with a hydroxyl; iv) —NRₘRₙ with Rₘ and Rₙ, which may be identical or different, representing a hydrogen atom or a (C₁-C₆)alkyl radical optionally substituted with a hydroxyl or (C₁-C₂)alkoxy group or a cationic group of formulae (1), (2) and/or (2′) as defined previously, the said alkyl radicals possibly forming, with the nitrogen atom that bears them, an optionally aromatic, optionally substituted, saturated or unsaturated 5- or 7-membered heterocycle, optionally comprising another nitrogen or non-nitrogen heteroatom; v) aryl such as phenyl; and vi) benzyl;
n and o are integers equal to 1, 2 or 3;
   it being understood that formula (IX) comprises at least one cationic group of formulae (1), (2) and/or (2′) as defined previously.

As examples of dyes of formula (IX), mention may be made of the salt derivatives mentioned, for example, in the patents: U.S. Pat. No. 3,625,947, U.S. Pat. No. 4,508,900, U.S. Pat. No. 4,595,756, FR-A-1 557 945, FR-A-2 010 444 and FR 0602608.

h) the tetraazapentamethine dyes of formulae (X) and (X′):

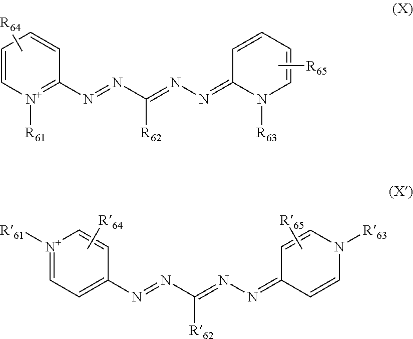

in which formulae (X) and (X′):
R₆₁, R₆₃, R′₆₁ and R′₆₃, which may be identical or different, represent a (C₁-C₈)alkyl radical optionally substituted with one to three radicals chosen from hydroxyl, (C₁-C₂)alkoxy, (poly)hydroxy(C₁-C₂)alkoxy, (di)(C₁-C₂)(alkyl)amino and carboxyl radicals;
R₆₂, R′₆₂, R₆₃, R′₆₃, R₆₄, R′₆₄, R₆₅ and R′₆₅, which may be identical or different, represent i) a hydrogen atom, or a radical chosen from ii) a linear or branched C₁-C₁₆ hydrocarbon-based chain, this chain possibly being saturated or unsaturated with one to three unsaturations, this chain being unsubstituted or substituted with one to three radicals chosen from hydroxyl, C₁-C₂ alkoxy, (poly)hydroxy(C₂-C₄)alkoxy, amino, (di)(C₁-C₂)alkylamino, carboxyl, sulfonylamino and (poly)hydroxy(C₂-C₄)alkylamino radicals or a halogen atom such as chlorine, fluorine or bromine; iii) phenyl optionally substituted with one to three radicals chosen from hydroxyl, C₁-C₂ alkoxy, (poly)hydroxy(C₂-C₄)alkoxy, amino, (di)(C₁-C₂)alkylamino, carboxyl, sulfonylamino and (poly)hydroxy(C₂-C₄)alkylamino radicals or a halogen atom such as chlorine, fluorine or bromine; and iv) a heteroaryl radical chosen from pyrazolyl, pyrrolyl, imidazolyl, thiazolyl, oxazolyl, triazolyl, pyridyl, pyrimidinyl, triazinyl, pyrazinyl and pyridazinyl radicals, furthermore this hydrocarbon-based chain may be interrupted with one or two oxygen, nitrogen or sulfur atoms or with a radical SO₂, it being understood that R₆₂, R′₆₂, R₆₃, R′₆₃, R₆₄, R′₆₄, R₆₅ and R′₆₅ do not comprise a peroxide bond or diazo or nitroso radicals.

As examples of dyes of formulae (X) and (X′), mention may be made of the salt derivatives mentioned, for example, in patent FR0753076.

i) the azo dyes of formula (XI):

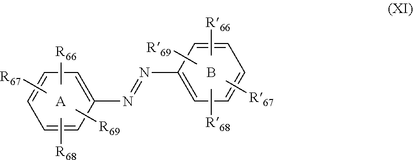

in which formula (XI):
R₆₆, R₆₇, R₆₈, R₆₉, R′₆₆, R′₆₇, R′₆₈ and R′₆₉, which may be identical or different, represent a hydrogen atom or a group chosen from:
   (C₁-C₆)alkyl;
   (C₁-C₆)alkoxy, (C₁-C₆)alkylthio;
   hydroxyl, mercapto;
   nitro;
   nitrile;
   R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom, or NR with R representing a hydrogen atom or an alkyl group;
   a cationic group of formulae (1), (2) and/or (2′) as defined previously;
   R″—S(O)₂—, with R″ representing a hydrogen atom or an alkyl, aryl, (di)(C₁-C₆)(alkyl)amino or aryl(C₁-C₆)(alkyl)amino group; preferentially a phenylamino or phenyl group;
   R′″—S(O)₂—X′— with R′″ representing a (C₁-C₆)alkyl group, optionally substituted aryl, X′ as defined previously;
   (di)(C₁-C₆)(alkyl)amino;
   aryl(C₁-C₆)(alkyl)amino optionally substituted with one or more groups chosen from i) nitro; ii) nitroso; iii) a cationic group of formulae (1), (2) and/or (2′) and iv) (C₁-C₆)alkoxy;
   optionally substituted heteroaryl; preferentially a benzothiazolyl group;
   cycloalkyl; especially cyclohexyl,
   Ar—N═N— with Ar representing an optionally substituted aryl group, preferentially a phenyl optionally substituted with one or more alkyl groups, cationic groups of formulae (1), (2) and/or (2′) or phenylamino groups;
   or alternatively two contiguous groups R₆₆ with R₆₇ or R₆₇ with R₆₈ or R₆₈ with R₆₉ together form a fused benzo group A′; and R′₆₆ with R′₆₇ or R′₆₇ with R′₆₈ or R′₆₈ with R′₆₉ together form a fused benzo group B′; with A′ and B′ optionally substituted with one or more groups chosen from i) nitro; ii) nitroso; iii) a cationic group of formulae (1), (2) and/or (2′) as defined previously; iv) hydroxyl; v) mercapto; vi) (di)(C₁-C₆)(alkyl)amino; vii) R^{o}—C(X)—X′—; viii) R^{o}—X′—C(X)—; ix) R^{o}—X′—C(X)—X″—; x) Ar—N═N— and xi) optionally substituted aryl(alkyl)amino; with R^{o}, X, X′, X″ and Ar as defined previously;
   it being understood that formula (XI) comprises at least one cationic radical of formulae (1), (2) and/or (2′) on one of the rings A or B.

As examples of dyes of formula (XI), mention may be made of the salts derived from: Basic Brown 16, Basic Brown 17, Basic Red 76, Janus Red, Basic Red 18, Basic Red 24, Basic Red 33.

j) the cyanine dyes of formula (XII):

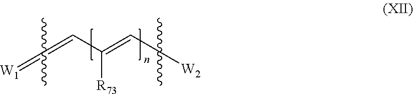

in which formula (XII):
W₁ represents a heteroaryl radical chosen from:

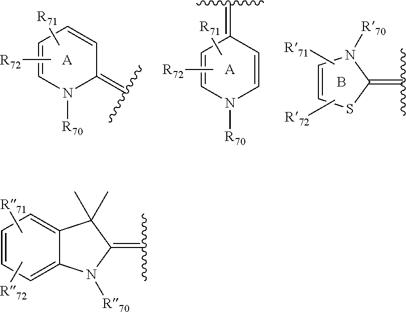

W₂ represents a heteroaryl radical bearing an endocyclic cationic charge chosen from:

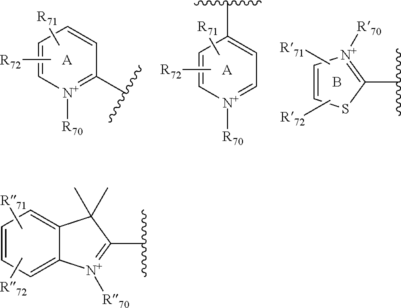

with:
R₇₀, R′₇₀, R″₇₀ and R₇₃, which may be identical or different, representing an optionally substituted C₁-C₂₀ alkyl radical, optionally interrupted with one or more heteroatoms and/or with one or more groups comprising at least one heteroatom, preferably chosen from oxygen, sulfur, C(O), S(O) and S(O)₂ or combinations thereof;
R₇₁, R′₇₁, R″₇₁, R₇₂, R′₇₂ and R″₇₂, which may be identical or different, representing i) an optionally substituted C₁-C₂₀ alkyl radical, optionally interrupted with one or more heteroatoms and/or with one or more groups comprising at least one heteroatom, preferably chosen from oxygen, sulfur, C(O), S(O) and S(O)₂ or combinations thereof; ii) (C₁-C₆)alkoxy; iii) (C₁-C₆)alkylthio; iv) hydroxyl; v) nitrile; vi) R^{o}—C(X)—X′—, R^{o}—X′—C(X)—, R^{o}—X′—C(X)—X″— with R^{o} representing a hydrogen atom or an alkyl or aryl group; X, X′ and X″, which may be identical or different, representing an oxygen or sulfur atom or NR with R representing a hydrogen atom or an alkyl group; vii) (di)(C₁-C₆)(alkyl)amino;
or alternatively two contiguous groups R₇₁ with R₇₂ together form a fused benzo group A; and R′₇₁ with R′₇₂ together form a fused benzo group B; with A′ and B′ optionally substituted with one or more groups chosen from i) alkyl; ii) hydroxyl; iii) alkoxy; iv) (di)(alkyl)amino; vi) R^{o}—C(X)—X′—; vii) R^{o}—X′—C(X)—; viii) R^{o}—X′—C(X)—X″—; with R^{o}, X, X′ and X″ as defined previously;
n is an integer between 0 and 3 inclusive.

As examples of dyes of formula (XII), mention may be made of the salts derived from: 1,3′-diethyl-4,2′-quinolylthiacyanine, 1,1′-diethyl-2,2′-cyanine, 3,3′-diethylthiacyanine, 5,5′-dimethoxy-3,3′-bis(3-sulfopropyl)thiacyanine, 3,3′-dipropylthiacarbocyanine, cryptocyanine, 3,3′-diethylthiacarbocyanine, 3,3′ diethyl-9-methylthiacarbocyanine, PIC, Cy2, Cy5, TO, Basic Red 12.

k) the phenoxazine, phenothiazine and phenazine dyes of formula (XIII):

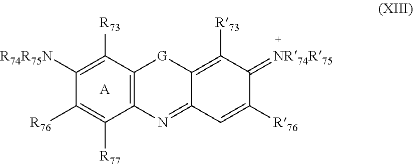

in which formula (XIII):
G represents an oxygen atom, a nitrogen atom or a radical NR₇₇;
—R₇₄, R₇₅, R′₇₄ and R′₇₅, which may be identical or different, represent an optionally substituted C₁-C₂₀ alkyl radical, optionally interrupted with one or more heteroatoms and/or with one or more groups comprising at least one heteroatom, preferably chosen from oxygen, sulfur, C(O), S(O) and S(O)₂ or combinations thereof;
R₇₇ represents an optionally substituted phenyl radical;
R₇₃, R₇₆, R′₇₃ and R′₇₆, which may be identical or different, represent a hydrogen atom, a C₁-C₂ alkyl radical or a halogen atom;
or alternatively two contiguous groups R₇₆ with R₇₇ together form a fused benzo group A.

As examples of dyes of formula (XIII), mention may be made of the salts derived from: Basic Blue 17, Basic Red 2, Basic Blue 12, Basic Blue 3, Basic Blue 9, Basic Violet 8.

More particularly, the dyes of formulae (II) to (XIII) that are useful in the invention are chosen from the salts derived from the following dyes:

| | | | |
|---|---|---|---|
| (C.I. 12251) | Basic Brown 17 | (C.I. 12250) | Basic Brown |
| | | | 16 |
| (CI 12719) | Basic Yellow 57 | Lowacryl Blue 9 | Basic Blue 9 |
| Lowacryl Blue 26 | Basic Blue 26 | Lowacryl Blue 7 | Basic Blue 7 |
| C.I. 11154 | Basic Blue 41 | Lowacryl Green 4 | Basic Green 4 |
| Lowacryl | Basic Orange 1 | Lowacryl | Basic Orange 2 |
| Orange 1 | | Orange 2 | |
| Vibracolor Flame | Basic Orange 31 | C.I. 45160 | Basic Red 1 |
| Orange | | | |
| Lowacryl Red 2 | Basic Red 2 | Lowacryl Red 22 | Basic Red 22 |
| Lowacryl Red 46 | Basic Red 46 | Vibracolor | Basic Red 51 |
| | | Ruby Red | |
| Lowacryl Violet 1 | Basic Violet 1 | Lowacryl Violet 2 | Basic Violet 2 |
| C.I. 42555 | Basic Violet 3 | Lowacryl Violet 4 | Basic Violet 4 |
| Rose B W 3005 | Basic Violet 10 | Lowacryl | Basic Violet 14 |
| (LCW) | | Violet 14 | |
| Lowacryl | Basic Yellow 11 | Lowacryl | Basic Yellow |
| Yellow 11 | | Yellow 28 | 28 |
| Vibracolor Citrus | Basic Yellow 87 | (C.I. 12245) | Basic Red 76 |
| Yellow | | | |

Imexidine BD or 1-(N-methylmorpholiniumpropylamino)-4-hydroxyanthraquinone

and the salt derivatives of formulae (V) and (V′) mentioned, for example, in patent applications FR0603322, FR0754454 and FR0858801; and of formulae (X) and (X′) mentioned, for example, in patent FR 0753076.

Most of these dyes are described in particular in the Color Index published by The Society of Dyers and Colorists, P.O. Box 244, Perkin House, 82 Grattan Road, Bradford, Yorkshire, BD12 JBN England.

The cationic dyes according to the invention may be obtained by exchanging the anionic counterion with one or more anionic counterions of the type X⁻ with X⁻ as defined previously.

Examples that may be mentioned include the following anionic dyes:

| Cationic part | |
|---|---|
| “derived from” a | |
| commercial dye | Corresponding structure |
| Basic Brown 17 | 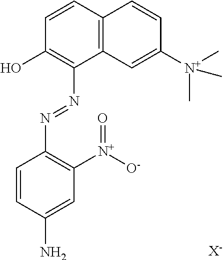 [SMILES: C[N+](C)(C)c1ccc2ccc(O)c(/N=N/c3ccc(N)cc3[N+](=O)[O-])c2c1 ; "X-"] |
| Basic Brown 16 | 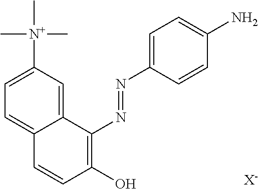 [SMILES: C[N+](C)(C)c1ccc2ccc(O)c(/N=N/c3ccc(N)cc3)c2c1 ; "X-"] |
| Basic Yellow 57 | 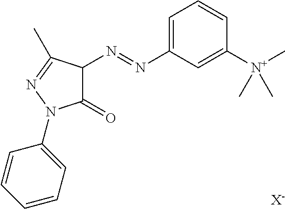 [SMILES: CC1=NN(c2ccccc2)C(=O)C1/N=N/c1cccc([N+](C)(C)C)c1 ; "X-"] |
| Imexine BD | 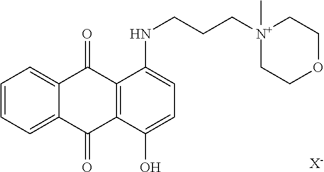 [SMILES: C[N+]1(CCCNc2ccc(O)c3c2C(=O)c2ccccc2C3=O)CCOCC1 ; "X-"] |
| Basic Red 51 | 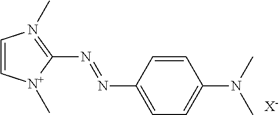 |
| Basic Yellow 87 | 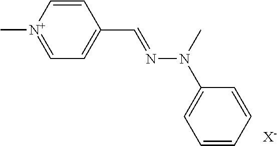 |
| Basic Orange | 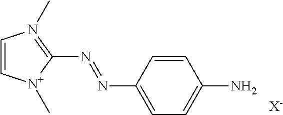 |

and the cationic salts of the dyes of formulae (V) and (V′) mentioned, for example, in patent applications FR0603322, FR0754454, FR0858801; of formulae (X) and (X′) mentioned, for example, in patent application FR 0753076 and of formulae (V) and (V′) mentioned, for example, in patent applications FR0603322, FR0754454, FR0858801 with X⁻ as defined previously.

According to preferred embodiment of the invention the cationic part of compounds of formula (I) is chosen from styryl dyes, preferably compounds (VIa) or (VI′a) as defined herein before and hydrazono dyes such as (V) and (V′) as defined herein before.

The dyes of formula (I) are derived from cationic dyes that are either commercially available or available via synthesis using standard synthetic techniques known to those skilled in the art. The “known” dyes comprise an anionic counterion, which is generally organic or mineral (chloride, methyl sulfate, etc.), which is replaced with a counterion X⁻ as defined previously. This substitution of anionic counterions may be performed via a standard ion-exchange method, for example by ion-exchange resin or ion-exchange column (ion-exchange methodology: see, for example, http://www.sigmaaldrich.com/analytical-chromatography/sample-preparation/spe/ionexchange-methodology.html and “Ion Exchange Material—Properties and Applications”, Andrei. A. Zagorodni, 1st Ed. 2007, Oxford, Elsevier BV; “Ion Exchange”, H. Friedrich G, 1995, NY: MacGraw-Hill, chapt. 2.3 p. 12: Ion Exchange Resins, chapt. 3, p. 29: Cation Exchangers; Chapt. 9, p. 421: Ion Exchange Column; Ullmann's Encyclopedia of Industrial Chemistry “Ion Exchange” F. Dardel and Thomas V. Arden, Published Online: Apr. 15, 2008, DOI: 10.1002/14356007.a14_{—}393.pub2; Kirk-Othmer Encyclopedia of Chemical Technology, “Ion Exchange” C. Dickert, Published Online: Dec. 4, 2000, DOI: 10.1002/0471238961.09151404090311.a01).

Another method consists in dissolving the known cationic dye in a water-immiscible organic solvent, such as halogenated organic solvents, for instance dichloromethane, chloroform or methyl tetrachloride, or aromatic organic solvents such as toluene, tetrahydrofuran (THF) or methyltetrahydrofuran (MeTHF), and in adding thereto salts of X⁻ in stoichiometric amount.

Depending on the added amount of salts of X⁻ in the organic solution and of the number of cationic groups, it is possible to replace one or more anionic counterions. If, for example, all of the anionic counterions must be replaced, then it is chosen to use a solution saturated with salt of X. The resulting mixture (salt of X⁻+ organic solvent+“known” cationic dye) is then left stirring at room temperature for between 1 minute and one week, such as from 30 minutes to 48 hours, in particular one day and preferentially between 2 and 4 hours. Next, the organic phase is filtered or evaporated, and then optionally washed with an alcoholic solution (ethanol, methanol, isopropanol, etc.) or aqueous solution (if the dye is not water-soluble) and separated again (by settling) or filtered. The organic phase is optionally dried using a standard drying agent such as alkali metal or alkaline-earth metal sulfate salts such as sodium sulfate, and is then filtered. The starting organic solvent is then evaporated off, for example using a rotary evaporator of Rotavapor® type.

### II. Composition Comprising at Least One Cationic Dye of Formula (I)

Another subject of the invention is a composition comprising, in a cosmetic medium, at least one cationic dye of formula (I) as defined previously, free of the dyes (A), (B), or even (C) and (D) as defined previously.

According to a particularly advantageous mode of the invention, the cosmetic composition comprising one or more dyes of formula (I) does not contain any chemical oxidizing agent.

The term “chemical oxidizing agent” means any chemical or enzymatic oxidizing agent other than atmospheric oxygen.

The dye composition that is useful in the invention generally contains an amount of cationic dye of formula (I) of between 0.001% and 50% relative to the total weight of the composition. Preferably, this amount is between 0.005% and 20% by weight and even more preferentially between 0.01% and 5% by weight relative to the total weight of the composition.

The dye composition may also contain additional direct dyes other than those of formula (I). These direct dyes are chosen, for example, from neutral, anionic or cationic nitrobenzene direct dyes, neutral, anionic or cationic azo direct dyes, tetraazapentamethine dyes, neutral, anionic or cationic quinone and in particular anthraquinone dyes, azine direct dyes, triarylmethane direct dyes, indoamine direct dyes and natural direct dyes.

Among the natural direct dyes, mention may be made of lawsone, juglone, alizarin, purpurin, carminic acid, kermesic acid, purpurogallin, protocatechaldehyde, indigo, isatin, curcumin, spinulosin and apigenidin. Use may also be made of extracts or decoctions comprising these natural dyes and in particular henna-based poultices or extracts.

The dye composition may contain one or more oxidation bases and/or one or more couplers conventionally used for the dyeing of keratin fibres.

Among the oxidation bases, mention may be made of para-phenylenediamines, bis(phenyl)alkylenediamines, para-aminophenols, bis-para-aminophenols, ortho-aminophenols and heterocyclic bases, and the addition salts thereof.

Among these couplers, mention may be made especially of meta-phenylenediamines, meta-aminophenols, meta-diphenols, naphthalene-based couplers and heterocyclic couplers, and the addition salts thereof.

The coupler(s) are each generally present in an amount of between 0.001% and 10% by weight and preferably between 0.005% and 6% by weight relative to the total weight of the dye composition.

The oxidation base(s) present in the dye composition are each generally present in an amount of between 0.001% and 10% by weight and preferably between 0.005% and 6% by weight relative to the total weight of the dye composition.

In general, the addition salts of the oxidation bases and couplers which may be used in the context of the invention are especially chosen from the salts of addition with an acid, such as the hydrochlorides, hydrobromides, sulfates, citrates, succinates, tartrates, lactates, tosylates, benzenesulfonates, phosphates and acetates, and the salts of addition with a base, such as alkali metal hydroxides, for instance sodium hydroxide, potassium hydroxide, ammonia, amines or alkanolamines.

The medium that is suitable for dyeing, also known as the dye support, is a cosmetic medium generally formed from water or a mixture of water and of at least one organic solvent. Examples of organic solvents that may be mentioned include C₁-C₄ lower alkanols, such as ethanol and isopropanol; polyols and polyol ethers, for instance 2-butoxyethanol, propylene glycol, propylene glycol monomethyl ether, diethylene glycol monoethyl ether and monomethyl ether, and also aromatic alcohols, for instance benzyl alcohol or phenoxyethanol, and mixtures thereof.

When they are present, the solvents are preferably present in proportions preferably of between 1% and 99% by weight approximately and even more preferentially between 5% and 95% by weight approximately relative to the total weight of the dye composition.

The dye composition may also contain various adjuvants conventionally used in hair dye compositions, such as anionic, cationic, nonionic, amphoteric or zwitterionic surfactants or mixtures thereof, anionic, cationic, nonionic, amphoteric or zwitterionic polymers or mixtures thereof, mineral or organic thickeners, and in particular anionic, cationic, nonionic and amphoteric polymeric associative thickeners, antioxidants, penetrants, sequestrants, fragrances, buffers, dispersants, conditioning agents, for instance volatile or non-volatile, modified or unmodified silicones such as amino silicones, film-forming agents, ceramides, preserving agents, opacifiers and conductive polymers.

The above adjuvants are generally present in an amount for each of them of between 0.01% and 20% by weight relative to the weight of the composition.

Needless to say, a person skilled in the art will take care to select this or these optional additional compound(s) such that the advantageous properties intrinsically associated with the dye composition in accordance with the invention are not, or are not substantially, adversely affected by the envisaged addition(s).

The pH of the dye composition is generally between 3 and 14 approximately, preferably between 4 and 11 approximately and more particularly between 5 and 10. It may be adjusted to the desired value by means of acidifying or basifying agents usually used in the dyeing of keratin fibres, or alternatively using standard buffer systems.

Among the acidifying agents that may be mentioned, for example, are mineral or organic acids such as those chosen from: i) hydrochloric acid HCl, ii) hydrobromic acid HBr, iii) sulfuric acid H₂SO₄, iv) alkylsulfonic acids: Alk-S(O)₂OH such as methanesulfonic acid and ethanesulfonic acid; v) arylsulfonic acids: Ar—S(O)₂OH such as benzenesulfonic acid and toluenesulfonic acid; vi) citric acid; vii) succinic acid; viii) tartaric acid; ix) lactic acid; x) alkoxysulfinic acids: Alk-O—S(O)OH such as methoxysulfinic acid and ethoxysulfinic acid; xi) aryloxysulfinic acids such as tolueneoxysulfinic acid and phenoxysulfinic acid; xii) phosphoric acid H₃PO₄; xiii) acetic acid CH₃COOH; xiv) triflic acid CF₃SO₃H; and xv) tetrafluoroboric acid HBF₄.

They are more particularly chosen from hydrochloric acid, orthophosphoric acid, sulfuric acid, carboxylic acids, for instance acetic acid, tartaric acid, citric acid or lactic acid, and sulfonic acids.

Among the basifying agents, examples that may be mentioned include mineral and organic bases, more particularly aqueous ammonia, alkali metal carbonates, alkanolamines, such as mono-, di- and triethanolamines and derivatives thereof, sodium hydroxide, potassium hydroxide and the compounds of formula (y) below:

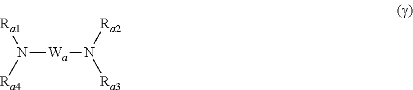

in which formula (y):
Wₐ is a (C₁-C₁₀)alkylene radical, optionally substituted with a hydroxyl group or a C₁-C₄ alkyl radical and/or optionally interrupted with one or more heteroatoms such as O or N; preferentially, Wₐ is a propylene;
Rₐi, Rₐ₂, Rₐ₃ and Rₐ₄, which may be identical or different, represent a hydrogen atom or a C₁-C₄ alkyl or C₁-C₄ hydroxyalkyl radical.

The dye composition may be in various forms, such as in the form of a liquid, a cream or gel, or in any other form that is suitable for dyeing keratin fibres, and especially the hair.

### III. Dyeing and Lightening Process Using a Cationic Dye of Formula (I)

Another subject of the invention is a process for dyeing keratin fibres, which consists in applying to the said fibres a composition comprising at least one cationic dye of formula (I) as defined previously.

The dyeing process according to the invention may also make it possible to optically lighten keratin fibres, especially dark keratin fibres such as keratin fibres with a tone depth of less than or equal to 6 and particularly less than or equal to 4, by using a composition comprising at least one fluorescent cationic dye of formula (I), which are preferentially dyes in the orange range. Mention may be made in particular of styryl or naphthalimide fluorescent dyes as defined previously, especially the derivatives present in patent application WO 03/028 685, and chosen especially from the dyes of formula (I) in which the cationic part of the direct dye is chosen from those of formulae (VI), (VIa), (VI′a), (VII), (Vila), (VIII), (VIII′), (VIII″), (VIII′″), (IX), (XII) and (XIII) as defined previously. According to a particularly advantageous mode, the fluorescent styryl dyes have the following formula:

W⁺—[C(R^{c})═C(R^{d})]_{m′}—Ar′ (VIa)

Ar—[C(R^{d})═C(R^{c})]_{m′}—W′⁺ (VI′a)

with (VI) and (VI′a) as defined previously and m=1.

According to a particularly advantageous embodiment of the invention, the dyeing or optical lightening process does not involve any chemical oxidizing agent.

According to one variant of the dyeing process, once the composition containing at least one cationic dye of formula (I) is applied to the keratin fibres, the composition is left on for a certain amount of time, and the keratin fibres are rinsed and/or drained dry and are then air-dried or dried using a hairdryer.

The duration of the treatment after application of the composition containing at least one dye of formula (I) may be short, for example from 0.1 second to 1 hour, particularly between 5 minutes and 50 minutes and more particularly between 10 and 45 minutes, and preferentially the leave-on time is 30 minutes.

The examples that follow serve to illustrate the invention without, however, being limiting in nature.

The cationic dyes in the examples hereinbelow were fully characterized by the standard spectroscopic and spectrometric methods.

### EXAMPLES

### Example of Preparation

General Preparation of the Dyes of the Invention: Counterion Exchange

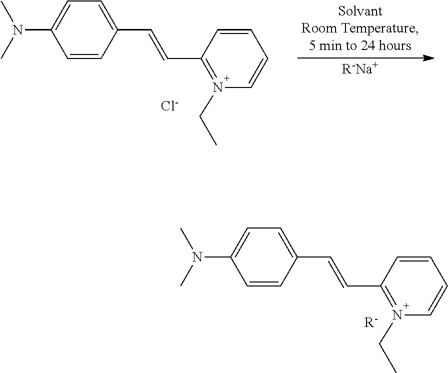

with R⁻ Na⁺ sodium hexanoate compound 1 and sodium dodecanoate compound 2 (*) such as dichloromethane, tetrahydrofuran, 2-methylTHF, diethyl ether, diisopropyl ether, heptane or toluene

Change of Halide Counterion for the Hexanoate Anionic Counterion

20 g of styryl dye are suspended in 600 ml of water-immiscible solvent* in the presence of 11.59 g of sodium hexanoate, in a conical flask. The reaction medium is stirred for 24 hours at room temperature and then filtered. The filtrate is then cleaned twice with isopropanol and then filtered again in order to remove the salts. The solvent is then evaporated off and the powder dried in a dessicator. The analysis performed confirm the structure of product 1 obtained.

Change of Halide Counterion for the Dodecanoate Anionic Counterion

20 g of styryl dye are suspended in 300 ml of water-immiscible organic solvent* in the presence of 18.66 g of sodium hexanoate, in a conical flask. The reaction medium is stirred for 24 hours at room temperature and then filtered. The filtrate is then cleaned twice with isopropanol and then filtered again in order to remove the salts. The solvent is then evaporated off and the powder dried in a dessicator. The analysis performed confirm the structure of product 2 obtained.

Preparation of Compound 1 to 5

General preparation of dyes according to the invention: exchanging of the counter-ion

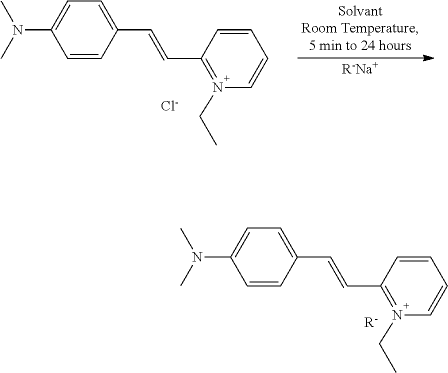

wherein R⁻ Na⁺ sodium hexanoate of compound 1, sodium dodecanoate of compound 2, sodium alginate of compound 3, sodium carboxymethylcellulose 4 and sodium sulphate laurylether of compound 5

(*) solvant such as dichloromethane; tetrahydrofurane, 2-methylTHF, diethylether, diisopropylic ether, heptane, toluene or benzene

Exchanging of Counter-Ion from Halide to Hexanoate

A styryl dye (20 g) is mixed at 600 ml of a water non miscible organic solvent* with sodium hexanoate (11.59 g) in a flask or erlenmeyer. Then the mixture is stirred for 24 hours at room temperature, and then filtered. The latter filtrate is then washed twice with isopropanol, then filtrated. Solvent is evaporated with vacuum and the crude powder is dried with P₂O₅ dessicator. The analysis performed are in accordance with the compound of formula 1.

Exchanging of Counter-Ion from Halide to Dodecanoate

A styryl dye (20 g) is mixed at 300 ml of a water non miscible organic solvent* with sodium dodecanoate (18.66 g) in a flask or erlenmeyer. Then the mixture is stirred for 24 hours at room temperature, and then filtered. The latter filtrate is then washed twice with isopropanol, then filtrated. Solvent is evaporated with vacuum and the crude powder is dried with a P₂O₅ dessicator. The analysis performed are in accordance with the compound of formula 2.

Exchanging of Counter-Ion from Halide to Laurylether Sulfate

A styryl dye (1.45 g) is mixed at 50 ml of a water non miscible organic solvent* with sodium alglinate (1 g) in a flask or erlenmeyer. A red precipate appeared. Then the mixture is stirred for 5 min at room temperature, and then filtered. The latter filtrate is then washed twice with isopropanol, then filtrated. Solvent is evaporated with vacuum and the crude powder is dried with a P₂O₅ dessicator. The analysis performed are in accordance with the compound of formula 3.

Exchanging of Counter-Ion from Halide to Anionic Carboxymethylcellulose

Sodium carboxymethycellulose (1 g) is solubilised in water (100 ml) at 80° C. After a complete solubilization, styrylic dye (1.62 g) is added to the mixture and stirred for 5 minutes at 80° C. The reaction mixture is then left it to cool at room temperature. A red gel is formed, filtered and the residue is then washed twice in water. The obtained gel is dried with a P₂O₅ desicator. The analysis performed are in accordance with the compound of formula 4.

Exchanging of Counter-Ion from Halide to Anionic Laurylethersulfate

A styryl dye (2.88 g) is mixed at 40 ml of water with sodium laurylethersulfate (2.88 g) in a flask or erlenmeyer, A red precipate appeared. Then the mixture is stirred for 15 min at room temperature, and the dark orange precipitate is then filtered. The latter filtrate is then washed twice with water, then filtrate. The latter is dried with a P₂O₅ dessicator. The analysis performed are in accordance with the compound of formula 5.

### Dyeing Hair Example

### Example 1

51 mg (1.26 10⁻⁴ mol) of compound 4 is solubilized in water (10 ml) with sonication in a warm bath (40° C.) for 1 hour. 2 ml of red solution is applied on one lock of keratin fibers (1 g, Caucasian White natural hair 90% and Japanese hair of tone height HT=2). After locks are rinsed with lukewarm water, and dried for 10 minutes. The styryl dye is applied under the same condition. Colorimetric measurement are realized with the aid of spectrocolorimeter Konica, 24 hours after dyeing treatment. Then, two successive shampoos are realized, and the locks of hair are dried.

After, the herein before treated locks are made moist with lukewarm water, 0.4 ml of shampoo is applied on each dyed locks, then locks are malaxed from top to bottom during 10 seconds then rinsed during 20 seconds with lukewarm water, and dried with hairdryer during 10 minutes. The latter shampoo/rinsed/dried process is repeated twice. Colorimetric measurements are realized with the same spectrocolorimeter as mentioned herein before, 24 hours after shampoo fastness evaluation.

Color Evaluation on Keratin Fibers

The colour of the hair was determined by using the L*a*b* system, with a Konica spectrophotometer.

According to this system, L* indicates the lightness. The lowest is the value of L*, the most intense is the color of the hair. The chromaticity coordinates are expressed by the parameters a* and b*, a* indicating the axis of red/green shades and b* the axis of yellow/blue shades.

The results are expressed in the following table.

ΔE, which is the color variation between a colored lock and a colored lock after shampoos, is obtained from the following formula:

ΔE=\sqrt{(L*−Lₒ*)²+(a*−aₒ*)²+(b*−bₒ*)²}

wherein L* indicates lightness and a* and b* are the chromaticity coordinates of the colored locks after successive shampoo whereas Lo* indicates the lightness and ao* et bo* are the chromaticity of the colored locks. The lowest is the value of ΔE, the most resistant is the color of the hair.

On Caucasian 90% White Natural Hair:

Composition containing compound 4 dyed hair in an intensive (L* data are significantly lower than the untreated hair (reference)) and chromatic orange attractive colour with a good shampoo fastness (ΔE=4.68)

TABLE 1

| | L* | a* | b* | ΔE |
|---|---|---|---|---|
| Before dyeing hair (reference) | 59.25 | 0.64 | 15.01 | — |
| After dyeing hair with compound 4 | 40.78 | 35.58 | 43.48 | 4.68 |
| After dyeing hair with compound 4 and | 43.32 | 37.76 | 46.75 | |
| successive shampoos | | | | |

On Japanese Brown Hair with Tone Height HT=2:

a) Lightening Effect of Cationic Dyes with Anionic Counter-Ion According to the Invention Vs. Same Cationic Dyes with Anionic Halide (Chloride, Out of the Invention)

ΔE*, is the color variation between a colored lock and a uncolored lock (reference) with compound of the invention, obtained from the following formula:

ΔE*=\sqrt{(L*−Lₒ*)²+(a*−aₒ*)²+(b*−bₒ*)²}

wherein L* indicates lightness and a* and b* are the chromaticity coordinates of the colored locks whereas Lo* indicates the lightness and ao* et bo* are the chromaticity of the uncolored locks. The highest is the value of ΔE*, the most visual lightening and chromatic effect is the color of the hair.

TABLE 2

| | L* | a* | b* | ΔE* |
|---|---|---|---|---|
| Before dyeing hair (reference) | 16.59 | 1.17 | 0.96 | 1.77 |
| After dyeing hair with dye out of the | 16.67 | 1.29 | 2.73 | |
| invention (halide) | | | | |

TABLE 3

| | L* | a* | b* | ΔE* |
|---|---|---|---|---|
| Before dyeing hair (reference) | 16.59 | 1.17 | 0.96 | 3.43 |
| After dyeing hair with compound 4 | 17.51 | 2.48 | 4 | |
| according to the invention | | | | |

Composition containing compound 4 lightened and dyed dark keratin fibers more efficiently than comparative compound bearing a chloride counter anion (see L data and ΔE* which are higher than untreated dark hair (reference)).

b) Lightening Fastness Effect Vs. Successive Shampoos ΔE

TABLE 4

| | L* | a* | b* | ΔE |
|---|---|---|---|---|
| After dyeing hair with compound 4 | 17.51 | 2.48 | 4 | 0.72 |
| After dyeing hair with compound 4 and | 18.17 | 2.21 | 3.85 | |
| successive shampoos | | | | |

Composition containing compound 4, lightened and dyed dark keratin fibers, with a good fastness vs. successive shampoos.

### Example 2

compound 3 (54 mg=1.26 10⁻⁴ mol) is solubilized and sonicated in warm water (10 ml, 40° C.) for 1 hour.

2 ml of red solution is applied on one lock of keratin fibers (1 g, Caucasian White natural hair 90% and Japanese hair with a tone height HT=2). After locks are rinsed with lukewarm water and dried for 10 minutes. The styryl dye is applied under the same condition. Colorimetric measurement are realized with the aid of spectrocolorimeter Konica, 24 hours after dyeing treatment. Then, two successive shampoos are realized, and locks are dried.

After, the herein before treated locks are made moist with lukewarm water, 0.4 ml of shampoo is applied on each dyed locks, then locks are malaxed from top to bottom during 10 seconds then rinsed during 20 seconds with lukewarm water, and dried with hairdryer during 10 minutes. The latter shampoo/rinsed/dried process is repeated twice. Colorimetric measurements are realized with the same spectrocolorimeter as mentioned herein before, 24 hours after shampoo fastness evaluation.

On Caucasian 90% White Natural Hair:

Composition containing compound 3 dyed in an intensive (L* data are significantly lower than the untreated hair (reference)) and chromatic orange hair with a good shampoo fastness (ΔE=4.78)

TABLE 5

| | L* | a* | b* | ΔE |
|---|---|---|---|---|
| Before dyeing hair (reference) | 59.25 | 0.64 | 15.01 | — |
| After dyeing hair with compound 3 | 44.67 | 33.16 | 49.32 | 4.78 |
| After dyeing hair with compound 3 and | 46.62 | 36.66 | 51.93 | |
| successive shampoos | | | | |

On Japanese Brown Hair with Tone Height HT=2:

a) Lightening Effect of Cationic Dyes with Anionic Counter-Ion According to the Invention Vs. Same Cationic Dyes with Anionic Halide (Chloride, Out of the Invention)

ΔE*, is the color variation as defined herein before.

TABLE 6

| | L* | a* | b* | ΔE* |
|---|---|---|---|---|
| Before dyeing hair (reference) | 16.59 | 1.17 | 0.96 | 3.07 |
| After dyeing hair with compound 3 | 17.30 | 2.26 | 3.74 | |
| according to the invention | | | | |

If we compare results of table 2 and table 6, it is observed that composition containing compound 3 lightened and dyed dark keratin fibers more efficiently than comparative compound bearing a chloride counter anion (see L data and ΔE* which are higher than untreated dark hair (reference)).

b) Lightening Fastness Effect Vs. Successive Shampoos ΔE

TABLE 7

| | L* | a* | b* | ΔE* |
|---|---|---|---|---|
| After dyeing hair with compound 3 | 17.30 | 2.26 | 3.74 | 0.57 |
| After dyeing hair with compound 3 and | 17.36 | 1.87 | 3.32 | |
| successive shampoos | | | | |

Composition containing compound 3 lightened and dyed dark keratin fibers, with a good fastness vs. successive shampoos.

### Example 3

Compound 5 (65 mg=1.26 10⁻⁴ mol) is solubilized in a mixture of benzylic alcool/ethanol/water (0.5 ml/1.5 ml/8 ml) and sonicated (10 ml, 40° C.) for 30 minutes. 2 ml of red solution is applied on one lock of keratin fibers (1 g of Japanese hair with a tone height HT=2). After dyeing, locks are rinsed with lukewarm water, and dried for 10 minutes. The styryl dye is applied under the same condition. Colorimetric measurement are realized with the aid of spectrocolorimeter Konica, 24 hours after dyeing treatment.

TABLE 8

| | L* | a* | b* | ΔE* |
|---|---|---|---|---|
| Before dyeing hair (reference) | 16.59 | 1.17 | 0.96 | 2.34 |
| After dyeing hair with compound 5 | 18.65 | 0.82 | 2.02 | |
| according to the invention | | | | |

If we compare results of table 2 and table 8, it is observed that composition containing compound 5 lightened and dyed dark keratin fibers more efficiently than comparative compound bearing a chloride counter anion (see L data and ΔE* which are higher than untreated dark hair (reference)).

1.-17. (canceled)

18. A method for treating keratin fibers, comprising applying to said keratin fibers a composition comprising:
at least one compound chosen from cationic dyes of formula (I) below:
Col⁽⁺⁾ₘ[X⁻]ₙ (I)
optical isomers, geometrical isomers, and solvates thereof;
wherein:
X⁻ represents a counterion independently chosen from:
i) Rₐ—CO₂⁻;
ii) sulfonates R_{b}—SO₃⁻;
iii) sulfates R_{c}—O—SO₃⁻;
iv) phosphates R_{d}—O—P(O)(OH)O⁻ or R_{d}—O—PO₃²⁻;
v) phosphate esters Rₑ—P(OR_{f})O₂;
vi) L-(X′⁻)ₓ;
vii) anionic oligomers comprising at least one anionic group X′⁻ other than vi);
viii) anionic polymers comprising at least one anionic group X′⁻ other than vi); and
ix) mixtures thereof;
wherein:
X′⁻ is independently chosen from groups CO₂⁻, SO₃⁻, O—SO₃⁻, O—PO₃²⁻ or P(OR_{f})O₂⁻;
x represents an integer ranging from 2 to 20 inclusive; and
L represents:
a) a linear or branched C₁-C₄₀ divalent or polyvalent hydrocarbon-based chain, optionally substituted, said chain being saturated or unsaturated, optionally interrupted and/or terminated at least one of its ends with one or more divalent groups chosen from:
—N(R^{a})—; —O—; —S—; —S(O)—, —S(O)₂—, —C(O)— and —C(S)— wherein R^{a} is chosen from a hydrogen atom and a C₁-C₄ alkyl, hydroxy(C₁-C₈)alkyl or amino(C₁-C₈)alkyl radical;
an aromatic or non-aromatic, saturated or unsaturated, fused or non-fused (hetero)cyclic radical, optionally comprising one or more identical or different, optionally substituted heteroatoms;
and combinations thereof;
b) an aromatic or non-aromatic, saturated or unsaturated, optionally substituted, optionally fused, divalent or polyvalent (hetero)cyclic group, optionally comprising one or more identical or different, optionally substituted heteroatoms;
Rₐ, R_{b} and R_{c} represent a radical chosen from:
i) optionally substituted linear or branched (C₄-C₄₀)alkyl;
ii) optionally substituted linear or branched (C₄-C₄₀)alkenyl;
iii) optionally substituted heteroaryl;
iv) optionally substituted (hetero)cycloalkyl;
v) optionally substituted linear or branched (hetero)aryl(C₁-C₄)alkyl;
vi) optionally substituted (hetero)cycloalkyl(C₁-C₄)alkyl;
the said alkyl or alkenyl group i) or ii) optionally interrupted and/or terminated with one or more identical or different divalent (hetero)aryls, divalent (hetero)cycloalkyls and heteroatoms chosen from an oxygen or sulfur atom, an amino, —N(R)—, —C(O)— or —C(S)— group or a combination thereof, with R representing a hydrogen atom or a (C₁-C₆)alkyl group;
Rₐ and R_{c} may also represent vii) an optionally substituted aryl group;
R_{d}, Rₑ and R_{f}, independently chosen from radicals:
i) optionally substituted linear or branched (C₁-C₄₀)alkyl;
ii) optionally substituted linear or branched (C₂-C₄₀)alkenyl;
iii) optionally substituted (hetero)aryl;
iv) optionally substituted (hetero)cycloalkyl;
v) optionally substituted linear or branched (hetero)aryl(C₁-C₄)alkyl;
vi) optionally substituted (hetero)cycloalkyl(C₁-C₄)alkyl;
the said alkyl or alkenyl group i) or ii) optionally interrupted and/or terminated with one or more identical or different divalent (hetero)aryls, divalent (hetero)cycloalkyls and heteroatoms chosen from an oxygen or sulfur atom, an amino —N(R)—, —C(O)— or —C(S)— group or a combination thereof, with R representing a hydrogen atom or a (C₁-C₆)alkyl group;
Col⁽⁺⁾ₘ represents the cationic part of a cationic direct dye comprising m cationic charge(s) comprising:
at least one heteroaryl bearing an endocyclic cationic charge, and/or
at least one (hetero)aryl or (hetero)cycloalkyl group bearing an exocyclic cationic charge;
the said cationic charges being chosen from:
a) the ammoniums of formula (1) below: 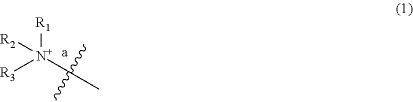
b) optionally substituted heteroaromatic groups bearing exocyclic or endocyclic, preferably endocyclic, cationic charge(s), such as (benz)imidazolium, indolinium, (benzo)triazolium, (benzo)pyrylium and (benzo)pyridinium groups of formulae (2) and (2′) below: 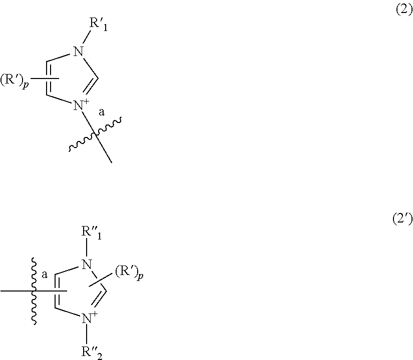
wherein, in formulae (1), (2) and (2′):
R₁, R₂ and R₃ are independently chosen from:
i) linear or branched (C₁-C₂₀)alkyl;
ii) (C₂-C₂₀)alkenyl;
iii) (hetero)aryl(C₁-C₂₀)alkyl, (hetero)cycloalkyl(C₁-C₂₀)alkyl or
iv) (hetero)aryl;
wherein the alkyl or alkenyl group of the groups of i), ii), iii) or iv) optionally are:
a) interrupted with one or more identical or different heteroatoms chosen from an oxygen or sulfur atom, an amino —N(R)—, ammonium —N⁺(Rₐ)(R_{b})—, —C(O)— or —C(S)— group or a combination thereof, with R, Rₐ and R_{b} independently chosen from hydrogen atoms or alkyl groups;
b) substituted with at least one hydroxyl, hydroxycarbonyl or carboxyl, (C₁-C₆)alkoxycarbonyl, alkyl(C₁-C₆)carbonyloxy, carbamoyloxy, (di)(C₁-C₆)(alkyl)silyl and tri(C₁-C₅)alkylsilyl group;
or alternatively the radicals R₁ and R₂ form, together with the quaternized nitrogen atom, a saturated 5- or 6-membered heterocycle, the said heterocycle being optionally partially unsaturated, optionally interrupted with a heteroatom chosen from the oxygen atom, the —N(R)— group, or ammonium —N⁺(Rₐ)(R_{b})— with R, Rₐ and R_{b} as defined previously and/or the said heterocycle optionally being substituted with one or more groups such as (C₁-C₆)alkyl;
R′₁, R″₁ and R″₂, which may be identical or different, represent a group chosen from i) linear or branched (C₁-C₂₀)alkyl; ii) (C₂-C₂₀)alkenyl; the alkyl or alkenyl group of the groups of i) and ii) optionally being interrupted with one or more identical or different heteroatoms chosen from an oxygen or sulfur atom or ammonium —N⁺(Rₐ)(R_{b})—, —C(O)—, —C(S)— or a combination thereof, with R, Rₐ and R_{b} as defined previously;
R′₁, R″₁ and R″₂, which may be substituted with at least one hydroxyl, hydroxycarbonyl or carboxyl, (C₁-C₆)alkoxycarbonyl, alkyl(C₁-C₆)carbonyloxy, carbamoyloxy, (di)(C₁-C₆)(alkyl)silyl or tri(C₁-C₅)alkylsilyl group;
R′ represents a group chosen from
i) (C₁-C₅)alkyl,
ii) (C₁-C₅)alkoxycarbonyl and
iii) hydroxyl;
a represents the linker arm connecting the cationic groups of formulae (1), (2) and/or (2′) to the dye or alternatively to an optionally substituted alkyl radical;
p is 0, 1, 2 or 3, it being understood that when p is 2 or 3, the radicals R′ are identical or different, or two radicals borne by contiguous carbon atoms together form a fused (hetero)aryl group such as benzo;
and
m and n independently chosen from an integer between 1 and 50 inclusive;
wherein:
when the cationic part of the direct dye contains a cationic group intrinsic to the structure or a cationic group of formula (1), (2) or (2′), then m=n=1; and
when the cationic part of the cationic direct dye contains cationic groups other than the endocyclic cationic group, it is combined with one or more organic anionic counterion(s) X⁻ for affording electrical neutrality to formula (I);
with the proviso that the compound of formula (I) is other than compound (A) below: 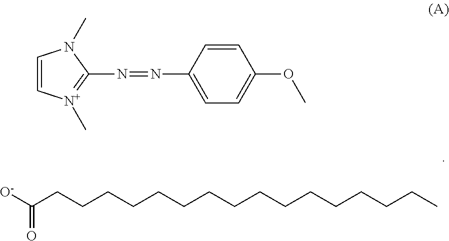 [SMILES: COc1ccc(N=Nc2n(C)cc[n+]2C)cc1 ; CCCCCCCCCCCCCCCCC(=O)[O-]]
